(19) United States
(12) Patent Application Publication
(10) Pub. No.: US 2024/0269338 A1
(43) Pub. Date: Aug. 15, 2024

(54) System and Method for Disinfection with Ultraviolet Light

(71) Applicants: John Johnston, Carmel, CA (US); BLUEMARBLE TECHNOLOGIES INC., Carmel, CA (US)

(72) Inventors: John Johnston, Carmel, CA (US); Subrata Dutta, Serilingampally (IN)

(21) Appl. No.: 18/568,311

(22) PCT Filed: Jun. 9, 2022
(86) PCT No.: PCT/US2022/032811

§ 371 Date: Dec. 8, 2023

(51) Int. Cl. A61L 9/20

(52) U.S. Cl. CPC A61L 9/20; A61L 2209/111; A61L 2209/16

(57) ABSTRACT

A system for at least partially disinfecting air in a duct. The system includes one or more light subassemblies having one or more light-emitting diodes configured to emit UVC light, and a control subassembly for energizing the light-emitting diodes. The control subassembly includes a diagnostics module for monitoring preselected parameters relating to the light subassembly, and a communications module for generating signals relating to the preselected parameters, and for transmitting the signals to one or more preselected recipients thereof. The system includes a display and a dashboard module for displaying data relating to the light subassemblies and the signals relating to the preselected parameters on the display. The system also includes sensors for monitoring air quality, and the air quality information is shown on the display. Suitable alarm signals are generated if the preselected parameters or if the air quality parameters are not within respective preselected acceptable ranges thereof.

### CROSS-REFERENCE TO RELATED APPLICATIONS

This application claims priority from U.S. Provisional Patent Application No. 63/208,866, filed on Jun. 9, 2021, and from U.S. Provisional Patent Application No. 63/278,275, filed on Nov. 11, 2021, the entire disclosures whereof are hereby incorporated by reference herein.

### FIELD OF THE INVENTION

The present invention is a system and a method for disinfection using ultraviolet light.

### BACKGROUND OF THE INVENTION

Ultraviolet germicidal irradiation is known, for inactivation of viruses and bacteria. In the prior art, low-pressure mercury vapor lamps are used to generate ultraviolet light, specifically UVC light, e.g., for disinfecting tools or surfaces. The ultraviolet light generated thereby has a wavelength of approximately 254 nm.

Specifically, low-pressure mercury lamps may be used to inactivate the novel Coronavirus, also referred to as Covid-19. The Centers for Disease Control and Prevention has defined “disinfection”, in part, as “a process that eliminates many or all pathogenic microorganisms . . . on inanimate objects . . . ”. Accordingly, when UVC is directed from the low-pressure mercury lamps toward inanimate objects to inactivate any infectious agents thereon, this may be described as disinfection.

There are some disadvantages to using low-pressure mercury lamps. First, the low-pressure mercury lamps are required to be energized continuously. Depending on the application in which they are utilized, this may result in a significant waste of electrical energy.

Second, the wavelength of the light emitted by the low-pressure mercury lamps is approximately 254 nm. However, it appears that, for inactivation of the novel Coronavirus, better results are achieved if the wavelength of the ultraviolet light is greater than 254 nm.

As is well known in the art, UVC light is harmful to humans. Accordingly, a device that emits UVC light that inactivates viruses and bacteria is required to be useable in a way that minimizes risk of human exposure to the UVC light that is generated.

### SUMMARY OF THE INVENTION

For the foregoing reasons, there is a need for a system and a method for disinfection utilizing ultraviolet light that overcomes or mitigates one or more of the defects or deficiencies of the prior art.

In its broad aspect, the invention provides a system for at least partially disinfecting air in a duct. The system includes one or more light subassemblies having one or more light-emitting diodes configured to emit UVC light, and a control subassembly for energizing the light-emitting diodes. The control subassembly includes a diagnostics module for monitoring preselected parameters relating to the light subassembly, and a communications module for generating signals relating to the preselected parameters, and for transmitting the signals to one or more preselected recipients thereof.

The system includes a display and a dashboard module for displaying data relating to the light subassemblies and the signals relating to the preselected parameters on the display. The system also includes sensors for monitoring air quality, and the air quality information may be shown on the display. Suitable alarm signals are generated if the preselected parameters or if the air quality parameters are not within respective preselected acceptable ranges thereof.

### BRIEF DESCRIPTION OF THE DRAWINGS

The invention will be better understood with reference to the attached drawings, in which:

FIG. 1A is an isometric view of an embodiment of a system of the invention;

FIG. 1B is a plan view of certain elements of the system of FIG. 1A, drawn at a larger scale;

FIG. 1C is a side view of an embodiment of the system of the invention installed in and on a duct housing, drawn at a smaller scale;

FIG. 2 is a side view of the system of FIG. 1A, drawn at a larger scale;

FIG. 3A is a side view of the system of FIG. 1A showing the system mounted inside a duct housing, drawn at a smaller scale;

FIG. 3B is a side view of a fastener securing a duct bracket of the system of FIG. 3A to an interior surface of the duct housing, drawn at a larger scale;

FIG. 4A is a cross-section of a duct housing in which a light subassembly of another embodiment of the system is mounted, drawn at a smaller scale;

FIG. 4B is a cross-section of another duct housing in which a light subassembly of an embodiment of the system is mounted;

FIG. 4C is a partial longitudinal section of the duct housing of FIG. 4A, showing the power enclosure box of the embodiment of the system;

FIG. 4D is a cross-section of another duct housing in which a light subassembly of an embodiment of the system is mounted;

FIG. 5A is a cross-section of another duct housing in which two light subassemblies of another embodiment of the system are mounted;

FIG. 5B is a cross-section of another duct housing in which two light subassemblies of another embodiment of the system are mounted;

FIG. 5C is a partial longitudinal section of the duct housing of FIG. 5A, showing the power enclosure box of the embodiment of the system;

FIG. 6A is an isometric view of another embodiment of a light subassembly in an alternative embodiment of the system, drawn at a larger scale;

FIG. 6B is an isometric view of the light subassembly of FIG. 6A mounted inside a duct housing, including a power enclosure box of the embodiment of the system, drawn at a smaller scale;

FIG. 6C is a cross-section of a duct housing in which multiple light subassemblies are mounted, across the duct housing;

FIG. 7A is an isometric view of an embodiment of a system of the invention mounted to a side wall of the duct housing, drawn at a smaller scale;

FIG. 7B is another isometric view of a light subassembly included in the system of FIG. 7A;

FIG. 7C is a top view of the system of FIGS. 7A and 7B mounted to the side wall of the duct housing, drawn at a smaller scale;

FIG. 7D is a side view of the system of FIGS. 7A-7C;

FIG. 8A is an isometric view of another embodiment of a light subassembly mounted to a side wall of the duct housing, drawn at a larger scale;

FIG. 8B is an isometric view of an embodiment of a system of the invention that includes the light subassembly of FIG. 8A;

FIG. 8C is a top view of the system and the duct housing of FIGS. 8A and 8B, drawn at a smaller scale; and

FIG. 8D is a side view of the system and the duct housing of FIGS. 8A-8C.

FIG. 9A is an elevation view of an alternative embodiment of the system of the invention positioned in a duct housing, drawn at a smaller scale;

FIG. 9B is a side view of the system of FIG. 7A showing the position thereof in a duct housing in relation to cooling coils of an air conditioning unit, drawn at a smaller scale;

FIG. 9C is an elevation view of another alternative embodiment of the system of the invention positioned in a duct housing, drawn at a smaller scale;

FIG. 10A is a diagram of a simplified circuit of an embodiment of a light subassembly of the invention including a voltmeter;

FIG. 10B is a block diagram schematically representing an embodiment of a fault monitor subsystem of the invention;

FIG. 11A is a side cross-section of an air duct with an embodiment of the system of the invention mounted in the air duct, drawn at a larger scale;

FIG. 11B is a top view of the air duct and the system of FIG. 11A;

FIG. 11C is an end view of the system of FIGS. 11A and 11B, drawn at a larger scale;

FIG. 11D is a side cross-section of an air duct with another embodiment of the light system of the invention mounted in the air duct, drawn at a smaller scale;

FIG. 11E is a top view of the air duct and the system of FIG. 11D;

FIG. 12A is a front view of another embodiment of the light subassembly of the invention, drawn at a smaller scale;

FIG. 12B is a side view of the light subassembly of FIG. 12A;

FIG. 12C is a side view of the light subassembly of FIGS. 12A and 12B mounted in the duct, drawn at a larger scale;

FIG. 13A is a front view of another embodiment of the light subassembly of the invention, drawn at a smaller scale;

FIG. 13B is a side view of the light subassembly of FIG. 13A;

FIG. 13C is a side view of the light subassembly of FIGS. 13A and 13B mounted in the duct, drawn at a larger scale;

FIG. 14 is a schematic illustration of a power enclosure box and two of the light subassemblies of FIG. 13A;

FIG. 15A is a schematic illustration of an exemplary display with data from the dashboard displayed thereon;

FIG. 15B is a schematic illustration of another exemplary display in which other data from the dashboard is displayed thereon; and

FIG. 16 is a schematic representation of an exemplary map of a facility in which a number of light subassemblies are installed.

### DETAILED DESCRIPTION

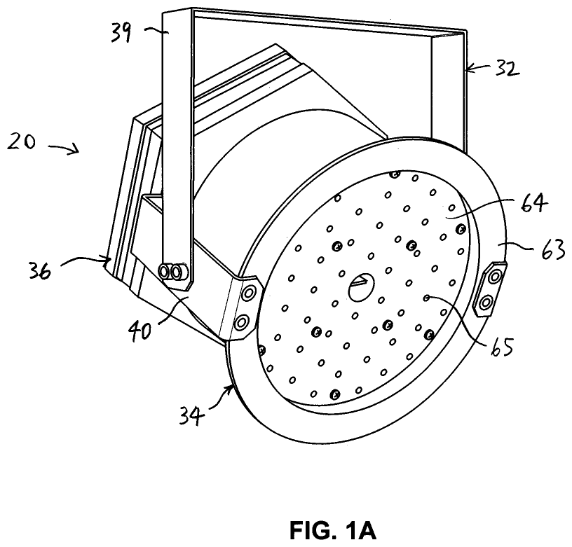

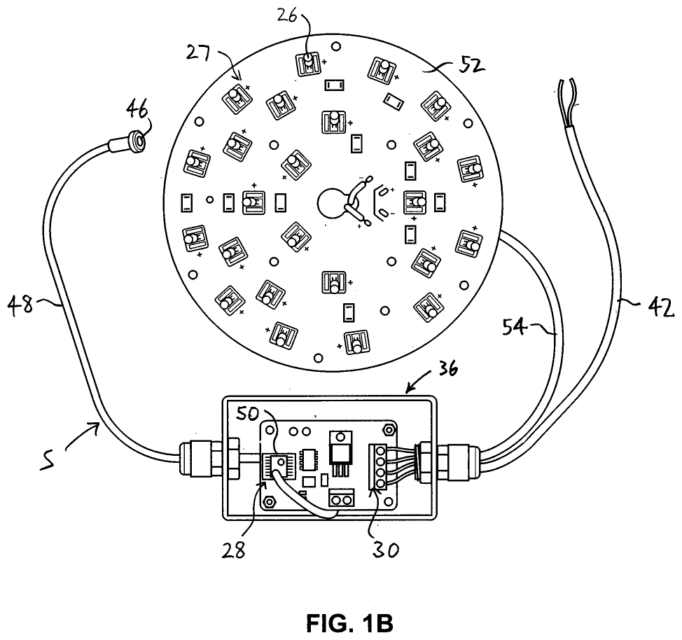

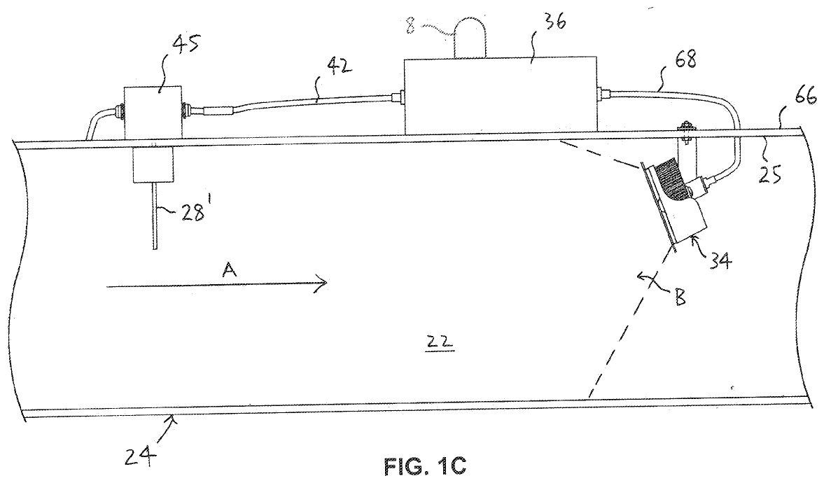

In the attached drawings, like reference numerals designate corresponding elements throughout. Reference is first made to FIGS. 1A-10B to describe an embodiment of a system in accordance with the invention indicated generally by the numeral 20.

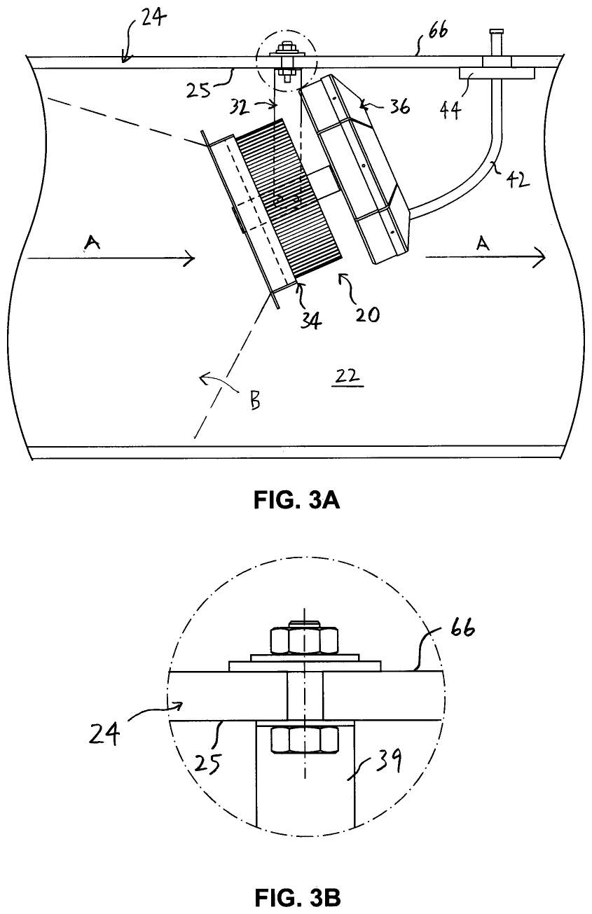

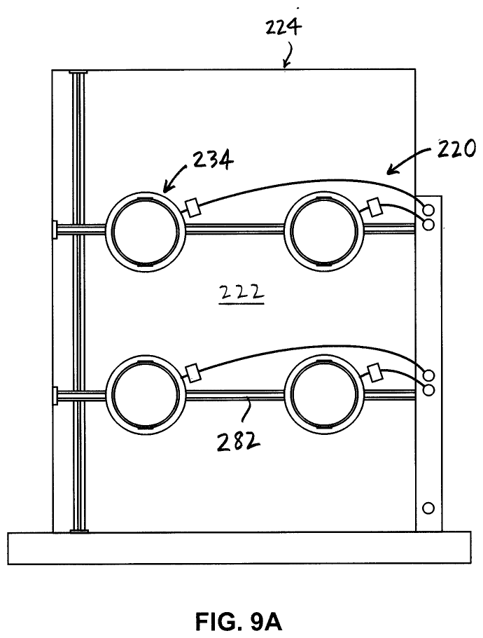

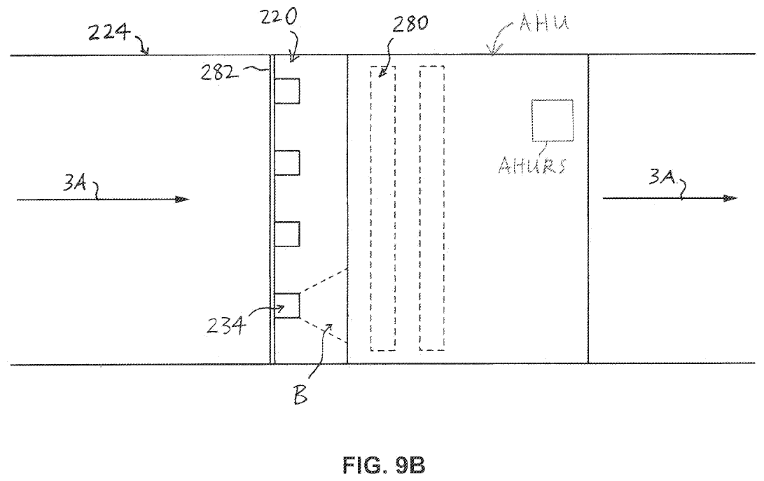

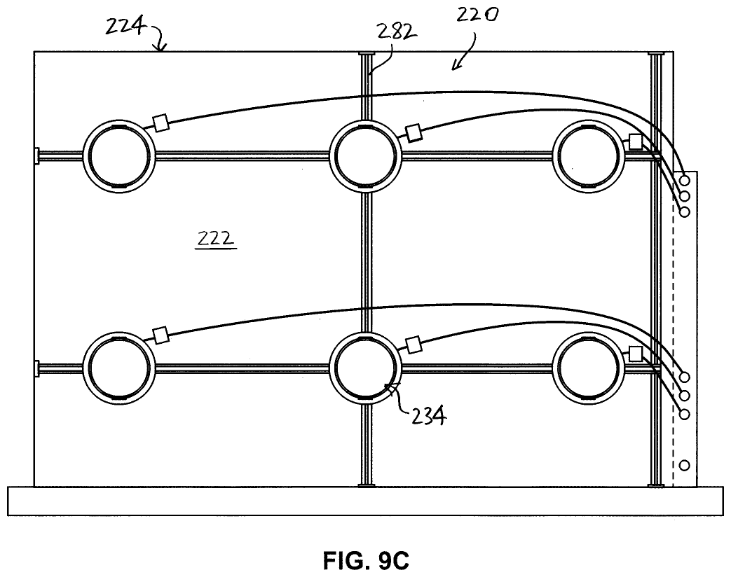

As will be described, the system 20 is for at least partially disinfecting air movable in a predetermined direction (FIG. 3A) through a duct 22 defined by a duct housing 24. The air is movable through the duct 22 by an air handling unit “AHU”, e.g., a conventional HVAC unit (FIG. 9B).

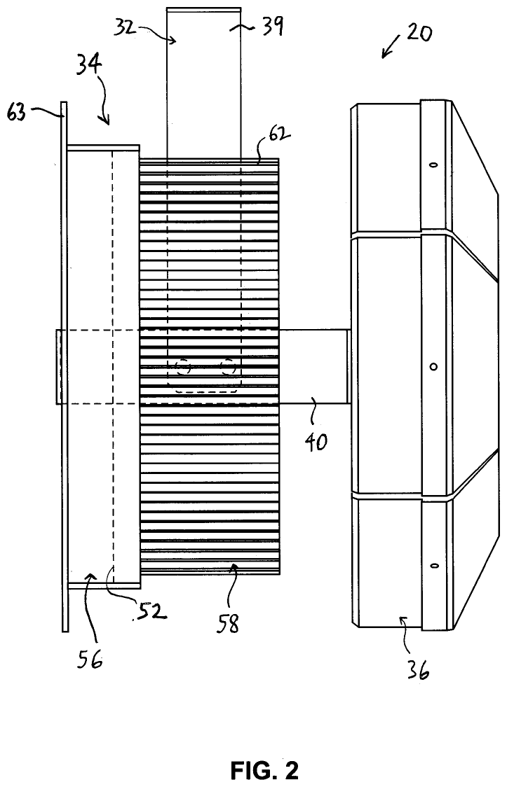

As can be seen in FIG. 3A, an interior surface 25 of the duct housing 24 defines the duct 22. In FIG. 3A, for example, the predetermined direction of air flow is indicated by arrow “A”. In one embodiment, the system 20 preferably includes one or more light-emitting diodes (LEDs) 26 (FIG. 2) configured for emitting ultraviolet light (UVC), for at least partially disinfecting the air. As will be described, the LEDs 26 may be arranged in an array 27 (FIGS. 1A, 1B).

Those skilled in the art would appreciate that UVC light may have wavelengths between 200 nanometers and 280 nanometers. Preferably, the LEDS 26 emit UVC light having a wavelength between approximately 241 nanometers and approximately 280 nanometers.

An embodiment of the system 20 is shown mounted inside the duct housing 24 in FIG. 3A, to the interior surface 25. As can be seen in FIGS. 1A, 1C, 2, and 3A, the system 20 preferably includes one or more light subassemblies 34 in which the LEDs 26 are mounted, a power enclosure box 36 inside which components of the control subassembly 30 are located, and a duct bracket subassembly 32. (It will be understood that part of the duct bracket subassembly 32 is omitted from FIG. 1C for clarity of illustration.)

Those skilled in the art would appreciate that, if required, a rectifier (not shown) may be located in the power enclosure box 36. However, those skilled in the art would appreciate that in certain circumstances, a direct DC feed may be provided, and a rectifier may then not be needed.

As will be described, in one embodiment, the system 20 may include a number of light subassemblies 34. For instance, a number of the light subassemblies 34 may be installed in a number of locations inside ducts throughout a commercial building, e.g., a grocery store, as will be described.

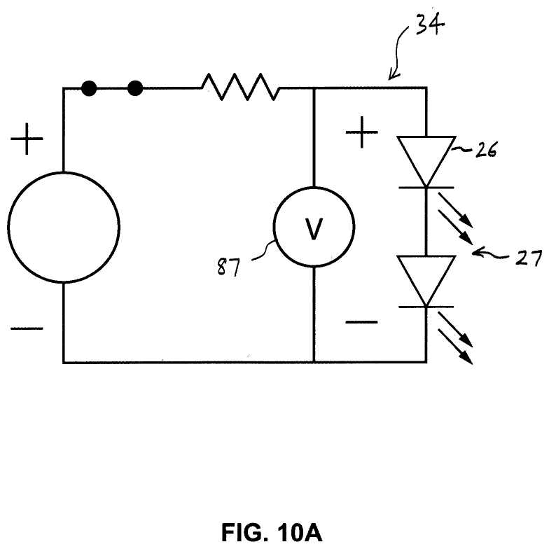

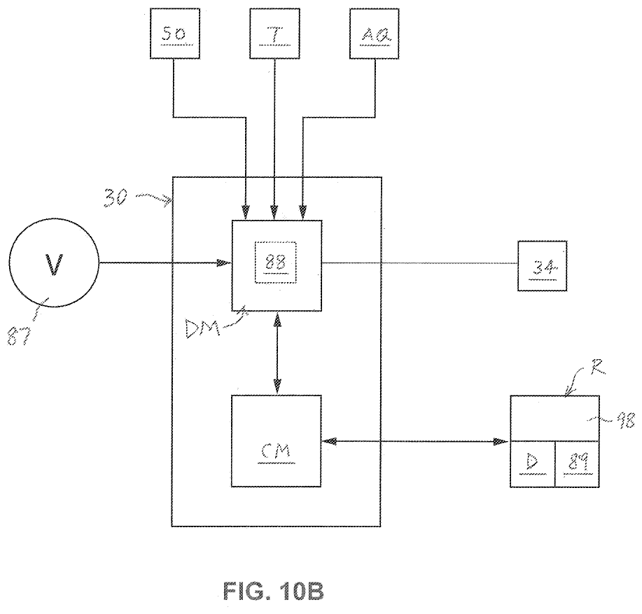

In one embodiment, the system 20 preferably also includes a control subassembly 30 (FIG. 1B), for energizing the LEDs 26. In one embodiment, the control subassembly 30 preferably includes a diagnostics module “DM” for monitoring preselected parameters relating to the light subassembly 34, and a communications module “CM” for generating signals relating to the preselected parameters, and for transmitting the signals to one or more preselected recipients “R” thereof (FIG. 10B).

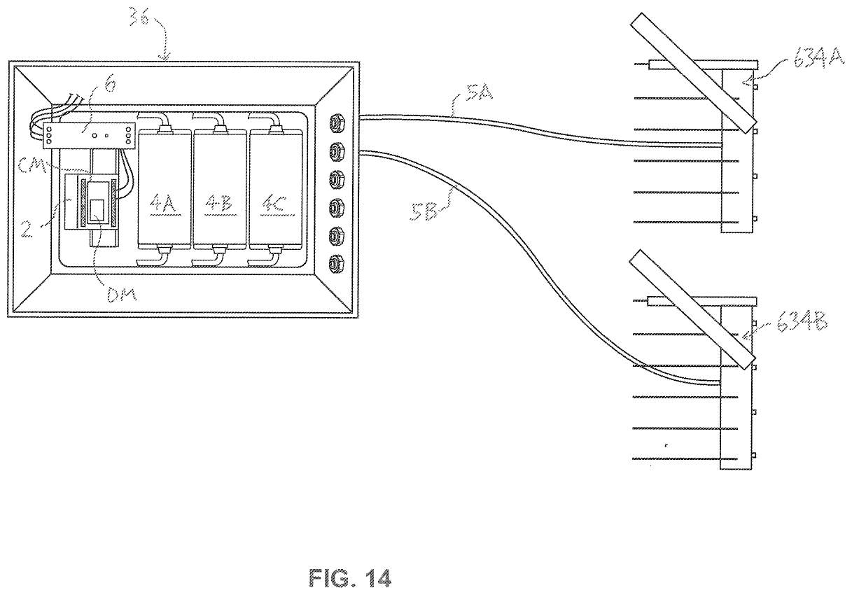

The diagnostics module or fault monitoring subsystem “DM” is for determining whether any one of the light subassemblies 34 is not functioning properly, i.e., is not within the predetermined parameters (FIGS. 10A, 10B). The diagnostics module “DM” monitors the light subassemblies 34 and detects fault conditions in the one or more light subassemblies 34 of the system. The diagnostics module “DM” is intended to provide an alert or message, via the communications module “CM”, if any one of the light subassemblies 34 is not working properly, so that steps may be taken promptly to rectify a failure or malfunction. The diagnostics module “DM” may disconnect the power source for any particular light subassembly 34 if the light subassembly 34 is not working properly, via a breaker 2 (FIG. 14).

An embodiment of the power enclosure box 36 is illustrated in FIG. 14, connected with two light subassemblies. For convenience, the two light subassemblies are identified by reference characters 634A and 634B. As illustrated in FIG. 14, three separate power supply units (identified in FIG. 14 by reference characters 4A, 4B, and 4C) are located inside the power enclosure box 36. Preferably, each of the power supply units 4A, 4B, 4C provides electrical power to a single light subassembly respectively. Power is provided to the light subassemblies 634A, 634B via power cords 5A, 5B respectively. The diagnostics module “DM” and the communications module “CM” are also located in the box 36 with a battery backup therefor, as is a power supply unit 6 for the diagnostics module “DM” and the communications module “CM”. It will be understood that the power supply units 4A, 4B, 4C, and 6 preferably are supplied with power from an external source, via conventional wiring (not shown in FIG. 14).

Those skilled in the art would appreciate that one diagnostics module “DM” may monitor the performance of a number of light subassemblies 34.

From the foregoing, it can be seen that the system 20 has the advantage that the UVC light emitted therefrom is emitted inside the duct housing 24. Because of this, in normal operating conditions, the system 20 can operate with practically no risk of exposure of humans to the UVC light generated thereby.

However, because the light subassembly 34 is intended to be installed so that the UVC light produced therefrom is contained within the duct housing 24, it is important to monitor the status of the light subassemblies 34, and the LEDs 26 therein, and to communicate information about the status thereof to the preselected recipients.

Accordingly, in one embodiment, the system 20 may include one or more alarm devices 8, for generating a suitable signal upon the diagnostics module “DM” detecting that the light subassembly 34 is not functioning properly, e.g., if the power supply is faulty, or if a LED circuit board is defective, or if one or more LEDs have failed. Preferably, the diagnostics module is configured (i) to determine whether a preselected parameter is within a predetermined acceptable range, and (ii) if the preselected parameter is outside the predetermined acceptable range, to generate and transmit an alarm signal to the alarm device.

The alarm device may be mounted, for example, to an exterior side of the duct housing 24, and/or it may be remotely located relative to the relevant light subassembly. The signal or signals provided by the alarm device may be any suitable signals. For instance, the alarm device may include one or more LEDs configured to generate light upon receipt of the alarm signal. In the example illustrated in FIG. 1C, the alarm device 8 includes an LED mounted to the power enclosure box 36. The alarm signal may also, or instead, be audible, if preferred. Alternatively, or in addition, the alarm device may generate suitable messages, to be sent by any suitable means, to alert the recipients that the system is not functioning properly.

Those skilled in the art would appreciate that various preselected parameters may be monitored to monitor the performance of a light subassembly. For instance, in one embodiment, the diagnostics module “DM” preferably determines whether the voltage applied to a selected one of the light subassemblies 34 is between predetermined upper and lower thresholds. An advantage of monitoring voltage is that it can be relatively simply done.

For example, in order to determine the voltage applied to a particular light subassembly 34, a suitable voltmeter 87 is connected to the light subassembly 34 (FIG. 10A). As a practical matter, it is preferred that the voltmeter 87 is a digital voltmeter. It will be understood that FIG. 10A is simplified for clarity, e.g., the array 27 is schematically indicated therein to include only two LEDs 26. The measured voltage data therefrom preferably is transmitted to a microprocessor 88 (FIG. 10B) of the diagnostics module that compares the measured voltage data to the predetermined upper and lower thresholds.

It will be understood that the microprocessor 88 preferably monitors the measured voltage on a substantially constant basis, e.g., the measured voltage data is updated at least once every few seconds. The microprocessor 88 preferably generates and transmits suitable signals, when the measured voltage data is outside the predetermined upper and lower thresholds, i.e., outside a predetermined acceptable range of voltages.

In one embodiment, the signals generated by the microprocessor 88 preferably are transmitted to the communications module “CM”, which may in response transmit suitable signals to the preselected recipients “R”. Preferably, the communications module “CM” utilizes any suitable technique(s) for transmitting outgoing signals to the preselected recipients “R”, and also for receiving incoming signals from the preselected recipients “R”.

Via the communications module “CM”, the microprocessor 88 of the diagnostics module is able to transmit to, and to receive from, communications with preselected recipients or contacts via any suitable means, whether wirelessly or via hardwire communications means. For instance, the communications module “CM” may utilize one or more of cellular communications technology, WiFi, or LoRa™. Upon receipt of the incoming signal, the communications module “CM” transmits the incoming signal to the microprocessor 88, where the incoming signal is processed.

Preferably, one of the preselected recipients is a dashboard module “D” configured for displaying data relating to the system on a dashboard on a display 89 (FIG. 10B), as will be described. The recipients “R” may also include a suitable device 98 operable by a human being (not shown) (FIG. 10B).

However, those skilled in the art would appreciate that the system 20 preferably also includes suitable relays or switches controllable by signals transmitted thereto from the diagnostics module “DM” by the microprocessor 88. As noted above, the breaker 2 (FIG. 14) may be controlled by the diagnostics module “DM”.

For example, if the measured voltage for a particular light subassembly 34 exceeds the predetermined upper threshold, then there is an overvoltage fault condition, which indicates that there is a fault in the LED array 27 or in the wiring in the light subassembly 34. Upon detection of an overvoltage condition, the microprocessor 88 generates a suitable overvoltage alert signal, to alert an operator, which is ultimately transmitted by the communications module “CM”, to the preselected recipients. Upon detecting the overvoltage, the microprocessor 88 preferably also transmits a disconnect signal, automatically causing the power supply to the light subassembly 34 to be disconnected.

If the measured voltage is less than the predetermined lower threshold, then there is an undervoltage fault condition, which indicates that there is a fault in the power supply to the light subassembly 34, or in the wiring in the light subassembly 34. Upon detection of an undervoltage fault condition, the microprocessor 88 generates a suitable undervoltage alert signal, ultimately transmitted by the communications module “CM” to the preselected recipients “R”, to alert the preselected recipients. Upon detecting the undervoltage, the microprocessor 88 preferably also sends a disconnect signal, automatically causing the power supply to the light subassembly 34 to be disconnected.

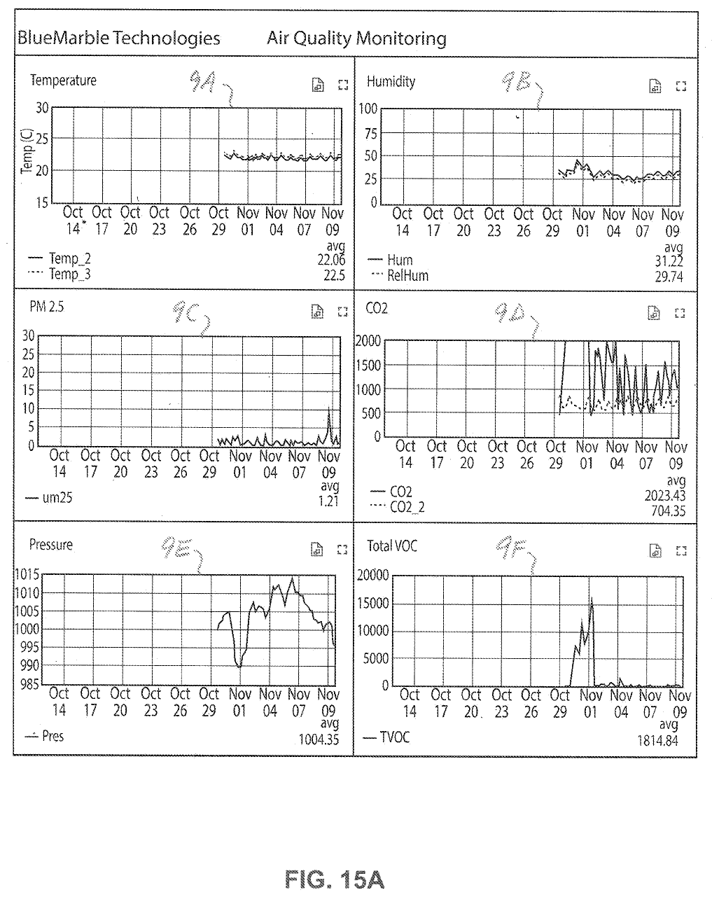

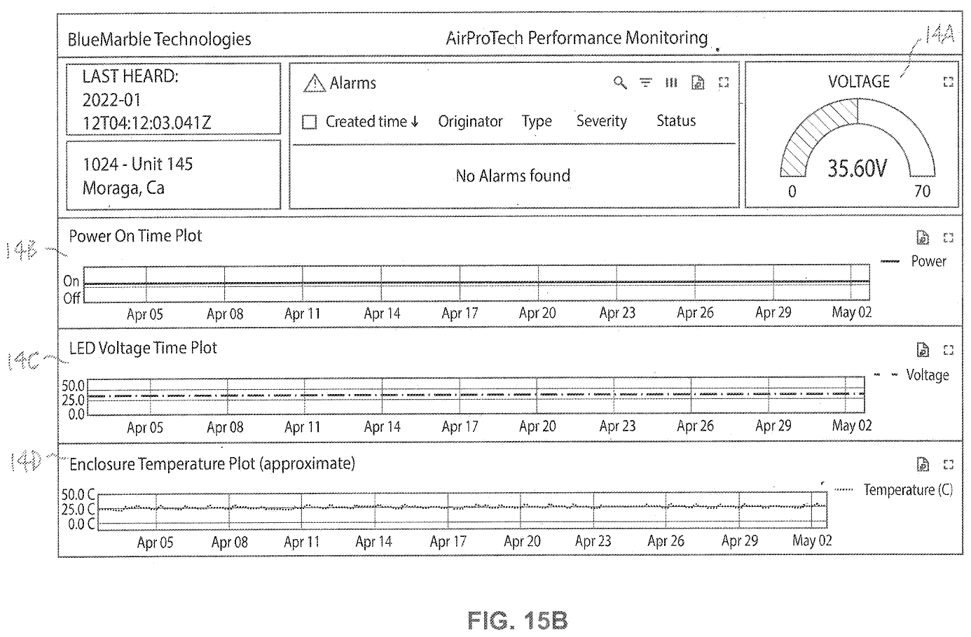

Preferably, the measured voltage data is provided on the display 89, in any suitable format, for the convenience of the operator (FIGS. 10B, 15B). In one embodiment, the system 20 preferably also includes the dashboard module “D”, for displaying data relating to the light subassemblies 34 of the system and signals relating to the preselected parameters thereof in a suitable format on the display 89.

Examples of information related to the monitored preselected parameters are provided in FIG. 15B. The information shown on the display 89 may be monitored by the operator to ensure that the system 20 as a whole is operating as intended. However, as noted above, if a measured preselected parameter is not within a predetermined acceptable range, a suitable signal (e.g., audible, visual, or otherwise) may be generated, and the diagnostics module “DM” may automatically disconnect one or more of the light subassemblies 34. In addition, upon the microprocessor 88 of the diagnostics module “DM” determining that one or more of the preselected parameters are not within the predetermined acceptable range therefor, suitable alarm signals or messages are communicated to the communications module “CM”, which are transmitted thereby to the preselected recipients “R”, including the dashboard module “D”.

As an example, in FIG. 15B, voltage is represented on a scale, identified by reference character 14A. Also shown in FIG. 15B are other examples of how measured data for preselected parameters may be presented on the display 89, e.g., a power on time plot, a LED voltage time plot, and an enclosure temperature plot, identified by reference characters 14B-14D respectively.

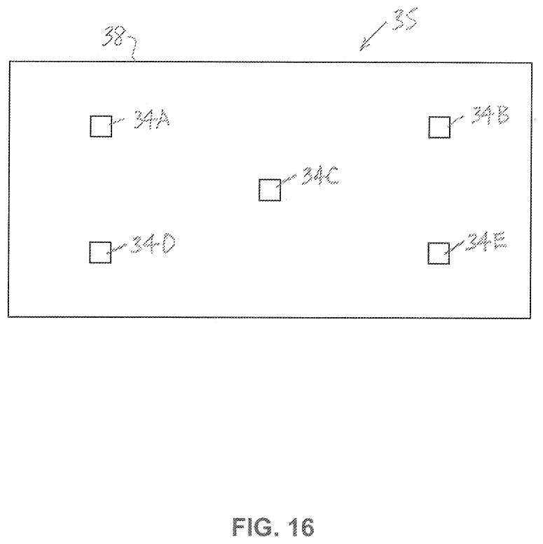

As noted above, the system 20 may include a number of light subassemblies, positioned at different locations throughout a building such as a grocery store or other large building. Accordingly, in one embodiment, the dashboard module “D” preferably also may generate a map 15 on the display 89, which enables the operator to identify the locations of the respective light subassemblies 34. An example of the map 35 is provided in FIG. 16, in which five light subassemblies identified by reference characters 34A-34E are shown, inside an outline 38 of the building. It will be understood that the map 35 generated by the dashboard “D” may include additional information (not shown in FIG. 16) to enable the operator to locate the light subassemblies.

Those skilled in the art would appreciate that, in the example noted above, the overvoltage and undervoltage alert signals may be provided in any suitable form(s), and sent via any suitable means. For example, text messages and/or e-mail messages may be transmitted to the devices 98 accessible by the preselected recipients “R”, who may then take appropriate action (FIG. 10B). Visual or audible signals also may be provided via the dashboard module “D”.

The diagnostics module “DM” described above is exemplary. It will be understood that, if desired, the diagnostics module “DM” may be configured to monitor preselected parameters other than overvoltage and undervoltage faults in the array 27 and in the light subassembly 34.

As noted above, one or more light subassemblies 34 may be de-activated (i.e., de-energized), when one or more of the preselected parameters for the light subassembly 34 are outside the predetermined acceptable range therefor. Or the system may be de-energized due to a general external power supply failure. The system 20 preferably is configured for subsequently re-activating the previously de-activated light subassemblies. In one embodiment, the re-activation preferably is initiated by transmitting a suitable reboot signal to the microprocessor 88. For instance, the reboot signal may be manually generated, so that the breaker 2 may be closed as a result. In this way, a part or all of the system may be remotely rebooted.

As is also noted above, the light subassemblies 34 may be positioned in the duct 22. As can be seen in FIGS. 1A, 2, and 3A, the duct bracket subassembly 32 is for locating the light subassembly 34 to direct the UVC light emitted from the LEDs 26 thereof into the air in the duct 22. The duct bracket subassembly 32 may include any suitable elements. As an example, the embodiment illustrated in FIGS. 1A, 2, and 3A preferably also includes a housing bracket 40 (FIG. 1A) to secure the power enclosure box 36 and the light subassembly 34 together. In one embodiment, the light subassembly 34 may consume 70 W.

In one embodiment, the duct bracket subassembly 32 preferably includes a duct bracket 39 that may be secured to the duct housing 24 by any suitable means. As can be seen in FIG. 3A, the light subassembly 34 preferably is mounted inside the duct housing 24 by securing the duct bracket 39 to the interior surface 25 of the duct housing 24. As shown in FIG. 3B, for example, the duct bracket 39 may be secured to the duct housing 24 by a bolt and a nut included in the duct bracket subassembly 32. The bolt may be inserted through a hole drilled in the duct housing 24. Preferably, the light subassembly 34 is positioned to direct the UVC light emitted by the LEDs 26 into the air (not shown) moving through the duct 22, in the direction indicated by arrow “A” so as to emit the light across the entire duct 22.

Those skilled in the art would appreciate that the light subassembly 34 may be retrofitted into an existing duct housing or mounted in a new duct housing, e.g., when the duct housing is installed.

From the foregoing, it can be seen that the light subassembly 34 may be mounted in a pre-existing duct housing relatively easily. First, part of the duct housing 24 may be cut or otherwise temporarily modified, to permit access to interior surfaces 25 of the duct housing 24. The bracket 39 may be mounted to the duct housing 24 in any suitable manner. For example, holes may be drilled in the duct housing 24, for mounting the bracket 39 to the duct housing 24. As will be described, another hole may be drilled through the duct housing 24, through which a power cord may be drawn.

Those skilled in the art would also appreciate that any opening(s) made in the duct housing 24 to permit installation of the system 20 preferably are closed and sealed, after the system 20 has been installed. However, it would be advantageous to be able to access the parts of the system 20 that are inside the duct housing 24 from time to time, after installation, and therefore it is preferred that the manner in which the opening is closed after installation should permit access to such parts of the system from time to time thereafter.

In new construction, the system may conveniently be installed in the duct housing before or after the duct housing is secured to the building.

The light subassembly 34 may be mounted to the duct housing 24 in any suitable manner. As will be described, some embodiments, the light subassembly 34 may not be mounted (via a bracket, or otherwise) to an inside surface of the duct housing, but instead may be secured to an exterior surface of the duct housing.

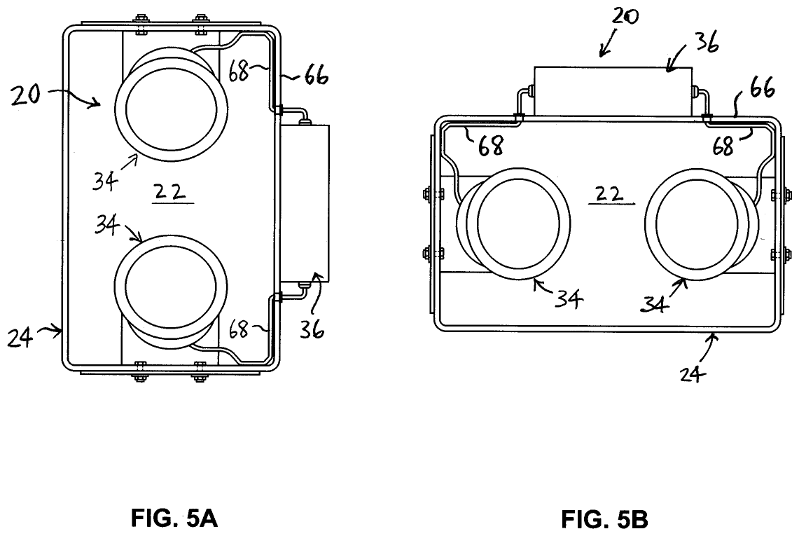

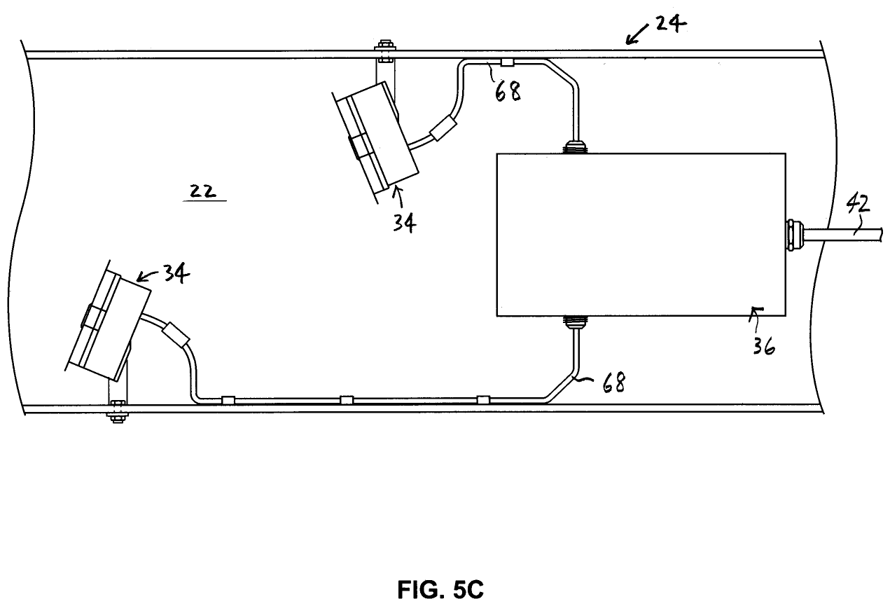

Those skilled in the art would also appreciate that any suitable power source may be utilized to provide electrical power to the light subassembly 34. As an example, in the embodiment illustrated in FIG. 3A, the power source is external. The connection with the power source may be provided by any suitable means. In one embodiment, electrical power preferably is provided via a power cord 42 that extends from the power enclosure box 36 to an external power source that is outside the duct housing 24 (FIG. 5C).

Those skilled in the art would appreciate that, in order to minimize energy consumption, the light subassemblies 34 preferably are respectively energized when the air is moving through the duct 22. Preferably, the control subassembly 30 is configured for energizing and de-energizing the LEDs 26 in direct relationship with movement of the air through the duct 24. Accordingly, in one embodiment, the system 20 preferably also includes an activation subassembly 28 for activating the control subassembly 30, to cause the control subassembly 30 to energize the LEDs 26, and for deactivating the control subassembly 30, to cause the control subassembly 30 to de-energize the LEDs 26. Preferably, the activation subassembly 28 causes energization of the LEDs 26 when the air is moving through the duct 22, and de-energization of the LEDs 26 when the air is not moving through the duct 22.

Because the LEDs 26 may be energized or de-energized depending on whether the air is flowing through the duct 24, the system 20 is energy-efficient.

In the example illustrated in FIG. 3A, a hole is drilled in the duct housing 24 in which a suitable grommet 44 is inserted, and the power cord 42 extends through the grommet 44 to connect the external power source with the power enclosure box 36. In one embodiment, the activation subassembly 28 preferably is partially located in the power enclosure box 36. It is also preferred that the control subassembly 30 in the power enclosure box 36 includes drivers and other components needed to enable the LEDs 26 to function.

In one embodiment, as can be seen in FIG. 1C, the system 20 may include a junction box 45, for AC feed and a switch controlled by the activation subassembly 28′.

In an alternative embodiment, the electrical power may be provided by one or more suitable batteries. However, those skilled in the art would appreciate that this arrangement may be suitable only if an external power source is not conveniently available.

Preferably, a number of the LEDs 26 are arranged in the array 27, positioned in a suitable pattern or arrangement, for directing the UVC light therefrom over a wide region inside the duct (FIGS. 1A, 1B). It will be understood that the array 27 illustrated in FIGS. 1A and 1B is only an example.

In the examples illustrated in FIGS. 1C and 3A, the UVC light emitted by the LEDs 26 has a beam angle of about 120°. Those skilled in the art would appreciate that the light beam angle may vary. The beam of UVC light collectively emitted from the LEDs 26 in the array 27 is schematically illustrated in FIGS. 1C and 3A, and identified by reference character “B” for convenience.

Those skilled in the art would appreciate that the LEDs 26 preferably are positioned so that the UVC light that is emitted thereby will affect all of the air moving through the duct 22 in the vicinity of the light subassembly 34, for inactivation of at least a portion of the infectious agents in the air moving through the duct 22, i.e., for at least partial disinfection of the air. It is believed that the UVC light inactivates a large portion of the infectious agents in the air moving past the light subassembly, and may inactivate all such infectious agents.

It is also believed that, depending on the size of the light subassembly 34, the cross-sectional size of the duct 22, and the rate at which the air flows through the duct 22, one light subassembly 34 may be sufficient for inactivation of at least a large portion of the infectious agents carried by the air moving through the duct 22. Alternatively, as will be described, depending on the cross-sectional area of the duct 22 and the rate of flow of the air, two or more light subassemblies 34 may be mounted at a location proximal to each other, to reduce the risk of failing to inactivate a portion of the infectious agents passing the location if only one subassembly 34 were used.

It is also believed that the UVC light is equally effective regardless of its direction into the air flow, relative to the predetermined direction of the air flow through the duct 22. The UVC light may be directed generally in upstream, downstream, or transverse directions (or combinations thereof) relative to the predetermined direction of the air flow in the duct 22.

As noted above, in one embodiment, due to the activation subassembly 28, the LEDs 26 preferably are energized only when air is flowing through the duct 22. Accordingly, in the embodiment illustrated, electrical energy is only consumed by the LEDs 26 when necessary, i.e., when air is directed through the duct 22, e.g., by one or more fans (not shown) in a HVAC system that includes the duct 22. The activation subassembly 28 may permit the LEDs 26 to be activated or energized, and de-activated or de-energized, in any suitable manner, depending on whether air is being drawn or forced through the duct 22, or not.

It will be understood that, in a commercial building, the air handling unit “AHU” may be a conventional HVAC unit, including heating and air conditioning portions thereof and a ventilation portion having one or more fans, for moving the air through the duct. Those skilled in the art would appreciate that the one or more fans of the air handling unit “AHU” is alternatively energized and de-energized via one or more relay switches “AHURS” (FIG. 9B) operatively connected with the air handling unit “AHU”. As is known in the art, the relay switch “AHURS” may be changed between an energizing condition, in which the one or more fans of the air handling unit are energized, and a de-energized condition, in which the one or more fans of the air handling unit are de-energized. Because conventional HVAC units are well known in the art, further description thereof is unnecessary.

In one embodiment, the activation subassembly 28 preferably is electrically connected with the relay switch “AHURS”. Preferably, the activation subassembly 28 causes the control subassembly 30 to energize the LEDs 26 upon energization of the one or more fans of the air handling unit via the relay switch “AHURS”, and the activation subassembly 28 causes the control subassembly 30 to de-energize the LEDs 26 upon de-energization of the one or more fans of the air handling unit via the relay switch “AHURS”.

For example, in one embodiment, electrical energy preferably is provided to the LEDs 26 via the power cord 42 only when the HVAC fans are energized. Those skilled in the art would appreciate that in this embodiment, a hardwired circuit (not shown) may connect the control subassembly 30 with the relay switch “AHURS” for the HVAC fans, so that activation of the fans of the HVAC system also causes the LEDs to be energized. In this embodiment, the power supply to the power enclosure box 36 is controlled by controlling the air handling unit “AHU” via the relay “AHURS” for the air handling unit. In this embodiment, when the fans of the HVAC system are de-activated (i.e., de-energized), the light subassembly 34 is also de-energized.

As will be described, other arrangements for energizing and de-energizing the light subassembly 34, based on whether air is flowing through the duct 22, may be utilized.

In one embodiment, the system 20 preferably includes one or more pressure sensors 50 positioned for detecting a pressure exerted by the air flowing in the duct 22. The pressure sensor 50 is configured to generate an activation signal when the pressure exerted by the air in the duct 22 is greater than a predetermined threshold activation pressure, the activation signal being transmitted to the control subassembly 30 for energization of the LEDs 26. Also, the pressure sensor 50 is configured to generate a deactivation signal when the pressure exerted by the air in the duct 22 is less than a predetermined threshold deactivation pressure, the deactivation signal being transmitted to the control subassembly 30 for de-energization of the LEDs 26.

As noted above, in one embodiment, the LEDs 26 preferably are energized when air is moving through the duct 22, and de-energized when air is not moving through the duct 22. The energization, and de-energization, preferably is controlled by the activation subassembly 28. In one embodiment, the activation subassembly 28 preferably includes a pressure-sensitive sensor subassembly “S” (FIG. 1B). The pressure-sensitive sensor subassembly “S” preferably includes an intake end 46 which is positioned inside the duct housing 24 to face upstream, relative to the air flow direction (FIG. 1B). Air flowing into the intake end 46 is directed through a connecting tube 48 to the pressure sensor 50 located inside the power enclosure box 36. When air flowing into the intake end 46 exerts a pressure greater than the predetermined threshold activation pressure, the pressure sensor 50 generates a signal transmitted to the control subassembly 30, to cause a switch or relay of the control subassembly 30 to close, causing electrical energy to be directed to the LEDs 26, energizing the LEDs 26 (FIG. 1B).

The switch or relay remains closed until the air pressure exerted by the air flow in the duct 22 is less than the predetermined threshold activation pressure. After activation, the system 20 is de-activated by a drop in pressure to a pressure that is less than the predetermined threshold activation pressure. When the air flowing into the intake end 46 exerts a pressure less than the predetermined threshold activation pressure, then the pressure sensor 50 generates a different signal that is transmitted to the control subassembly 30, to cause the switch or relay of the control subassembly 30 to open, thereby de-energizing the LEDs 26.

For instance, the predetermined threshold activation pressure may be the pressure exerted by the air moving through the duct 22 when the fans of the air handling unit “AHU” are energized.

In the embodiment illustrated in FIG. 1B, the activation subassembly 28 includes the pressure-sensitive sensor subassembly “S”, which includes the intake end 46, the connecting tube 48, and the pressure sensor 50.

It will be understood that various other types of activation subassemblies may be utilized. As noted above, in an alternative embodiment, energization and de-energization may be controlled via direct electrical connection of the activation subassembly 28 with the relay “AHURS” for the HVAC fans. As another example, the LEDs 26 may be energized based on changes in temperature of the air in the duct 22. This may vary with the seasons, e.g., in winter, the temperature sensors may cause activation when the air temperature is sufficiently low that the heater portion and the fans of the air handling unit are energized, and in summer, the temperature sensors may cause activation when the air temperature is sufficiently high that the air conditioning portion and the fans of the air handling unit are energized.

In one embodiment, the activation subassembly 28 preferably includes one or more temperature sensors “T” positioned for detecting a temperature of the air in the duct 22. The temperature sensor preferably is configured to generate an activation signal upon the temperature of the air in the duct 22 being greater than a first predetermined threshold summer temperature, the activation signal being transmitted to the control subassembly 30 for energization of the LEDs 26. The temperature sensor preferably also is configured to generate a deactivation signal upon the temperature of the air in the duct 22 being less than a second predetermined threshold summer temperature, the deactivation signal being transmitted to the control subassembly 30 for de-energization of the LEDs 26 (FIG. 10B).

Those skilled in the art would appreciate that the temperature sensor “T” may be located in any suitable location in the duct 22.

Those skilled in the art would also appreciate that the embodiment described above, in which the activation signal is generated when the air temperature in the duct 22 is higher than the first predetermined threshold summer temperature, may in practice be utilized generally during summer months. Preferably, the first predetermined threshold summer temperature is higher than the second predetermined threshold summer temperature. For example, the first predetermined threshold summer temperature may be 24° C., and the second predetermined threshold summer temperature may be 20° C. It is also preferred that the air handling unit “AHU” is configured to be energized and de-energized accordingly, so that both the air conditioning portion of the air handling unit and the LEDs 26 are activated when the temperature of the air in the duct 22 is higher than the first predetermined threshold summer temperature, and the air conditioning portion and the LEDs 26 are deactivated when the temperature of the air in the duct 22 is lower than the second predetermined threshold summer temperature.

In another embodiment, the activation subassembly 28 preferably includes one or more temperature sensors “T” positioned for detecting a temperature of the air in the duct 22. In this embodiment, the temperature sensor preferably is configured to generate an activation signal upon the temperature of the air in the duct 22 being less than a first predetermined threshold winter temperature, the activation signal being transmitted to the control subassembly 30 for energization of the LEDs 26. The temperature sensor preferably also is configured to generate a deactivation signal upon the temperature of the air in the duct 22 being greater than a second predetermined threshold winter temperature, the deactivation signal being transmitted to the control subassembly for de-energization of the LEDs 26.

Those skilled in the art would appreciate that the embodiment described above, in which the activation signal is generated when the air temperature in the duct 22 is lower than the first predetermined threshold winter temperature, may in practice be utilized generally during winter months. Preferably, the first predetermined threshold winter temperature is lower than the second predetermined threshold winter temperature. For example, the first predetermined threshold winter temperature may be 20° C., and the second predetermined threshold winter temperature may be 23° C. It is also preferred that the air handling unit “AHU” is configured to be energized and de-energized accordingly, so that both the heater portion of the air handling unit and the LEDs 26 are activated when the temperature of the air in the duct 22 is lower than the first predetermined threshold winter temperature, and the heater portion and the LEDs 26 are deactivated when the temperature of the air in the duct 22 is lower than the second predetermined threshold winter temperature.

In yet another embodiment, a timer may be used, to cause the LEDs to be energized for certain periods of time at predetermined intervals, regardless of the flow of air through the duct at any particular time. The intervals may be determined, based on air flow and the time periods when the fans are energized on average, so that all or substantially all of the air moved through the duct 22 is subjected to the UVC light over time.

Those skilled in the art would appreciate that, alternatively, the LEDs may simply be energized, for an indefinite period of time. In view of the relatively low power consumption of the light subassembly 34, this may be suitable in certain circumstances.

In FIG. 1C, the system 20 is shown as including an activation subassembly 28′ located upstream in relation to the light subassembly 34. It will be understood that the activation subassembly 28′ may include a pressure-sensitive sensor, as described above. Alternatively, the activation subassembly 28′ may include a temperature-sensitive sensor, or any other suitable sensor for activation and de-activation of the light subassembly 34 in relation to air flowing through the duct 22.

Once the LEDs 26 are energized, it is preferred that the control subassembly 30 causes each of the LEDs to be de-energized respectively for a predetermined “off” time period, after having been continuously energized for a predetermined “on” time period. As an example, the off time period may be 10 milliseconds, and the predetermined on time period may be two seconds. Preferably, only one LED, in an array that includes many LEDs, may be off at any one time, and while the selected LED is de-energized, all the other LEDs in the array 27 are energized. It is also preferred that the off time period is sufficiently short that, as a practical matter, the lack of UVC light emitted by the LED selected to be off in the off time period is insignificant (because of the UVC light emitted by the other LEDs in the array during the off time period), as far as the disinfection of the air is concerned. In order to minimize any adverse effects from the off time period, it is also preferred that the LEDs in the array 27 are de-energized during their respective off time periods at different times.

It is preferred that the control subassembly 30 is configured to control the energization of the LEDs to provide the predetermined off and on time periods described above, because this is believed to extend the life of the p-n junction in each respective LED. After each LED is de-energized for the preselected off time period respectively, each LED is respectively energized. As is known, over time, the quantum mechanical forces to which the imaginary thin wall at the p-n junction is subjected, during the forward biased condition, may cause gradual crystal deformation at the p-n junction, resulting in a shifting of the UVC spectrum and power. It is believed that causing the LED to be de-energized during the off time period and energized during the on time period as outlined above will mitigate, or possibly prevent, the gradual crystal deformation.

As noted above, because the respective LEDs in the array are off (de-energized) for only a short period of time during which the other LEDs in the array are energized, it is believed that the benefit of the short off period can be realized without a significant reduction in the disinfecting effectiveness of the array 27 as a whole.

In summary, in one embodiment, the control subassembly 30 preferably is configured for de-energizing at least one preselected one of the light-emitting diodes 26 in the array 27 for a preselected off time period while a balance of the array 27, which consists of the light-emitting diodes 26 other than the preselected one, is energized. Preferably, while the balance of the array 27 is energized, each of the light-emitting diodes 26 is de-energized respectively for the preselected off time period.

As can be seen in FIG. 1B, in one embodiment, the light subassembly 34 preferably includes a circuit board 52 on which the LEDs 26 are mounted. Preferably, an internal power cord 54 electrically connects the board 52 and the power cord 42 when the switch or relay of the control subassembly 30 is closed, for energizing the LEDs 26.

As can be seen in FIGS. 1A and 2, in one embodiment, the light subassembly 34 preferably includes a light housing 56 in which the board 52 is mounted (FIG. 2), with the array 27 of the LEDs 26 located thereon. The light housing 56 preferably includes a heat sink portion 58 formed for dissipating heat generated by the LEDs 26 when they are energized (FIG. 2). As shown in FIG. 2, the heat sink portion 58 preferably includes fins 62 for heat dissipation therefrom. (It will be understood that part of the housing bracket 40 is omitted from FIG. 2 for clarity of illustration.)

In one embodiment, the light housing 56 preferably includes a face plate 64 with holes 65 therein, the holes 65 being aligned with the LEDs 26 to allow the UVC light emitted by each of the LEDs 26 respectively to pass through the respective holes 65 in the plate 64 (FIG. 1A). The face plate 64 protects the LEDs 26. As can be seen in FIGS. 1A and 2, in one embodiment, the outer portion 60 preferably also includes an outer flange 63, to which the housing bracket 40 preferably is fixed. The duct bracket 39 may be secured to the housing bracket 40 (FIG. 1A).

As can be seen in FIGS. 1C and 4A-5C, the power enclosure box 36 may be mounted on an exterior surface 66 of the duct housing 24. These embodiments have the advantage that the power enclosure boxes 36 thereof are easy to access, so that service or replacement of the power enclosure boxes may be conveniently done without opening the duct housing 24.

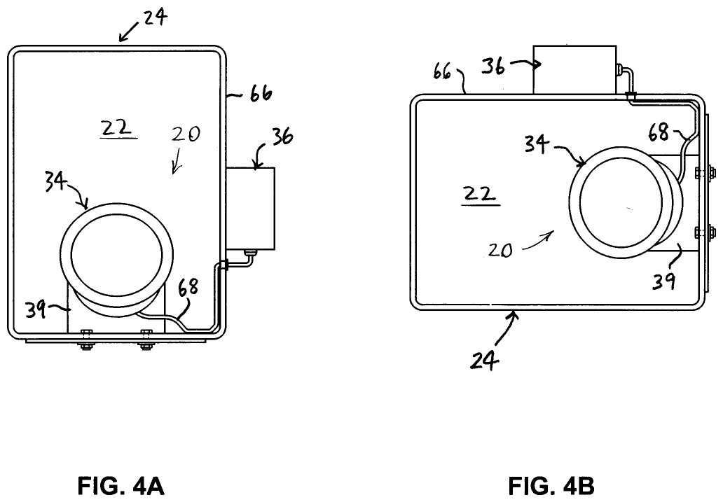

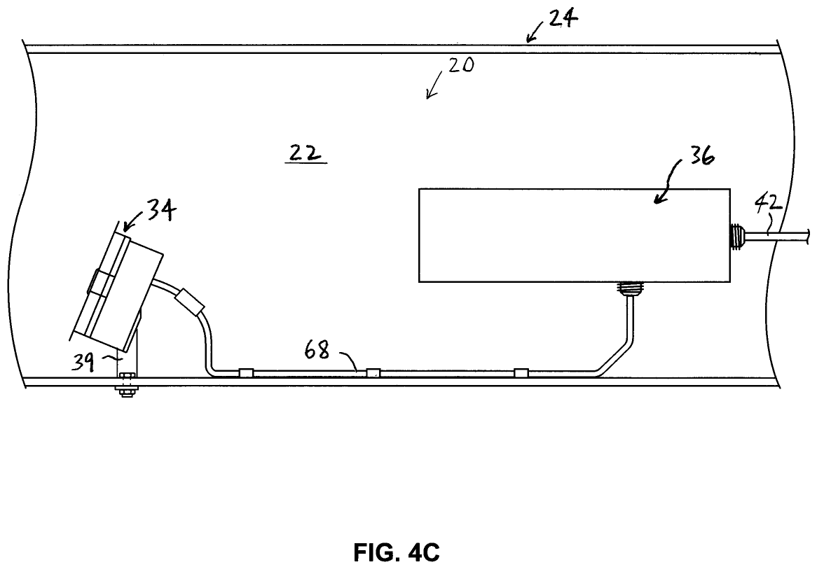

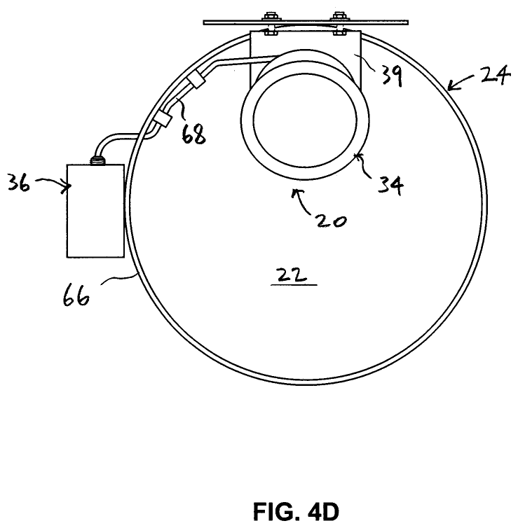

In these embodiments, an intermediate power cord 68 electrically connects the light subassembly 34 and the control subassembly 30 inside the power enclosure box 36. As can be seen, e.g., in FIGS. 1C and 4A, the power enclosure box 36 may conveniently be mounted to the exterior surface 66 of the duct housing 24, and the duct bracket 39 and the light subassembly 34 are located inside the duct housing 24. Those skilled in the art would appreciate that it is relatively easy to connect the power enclosure box 36 and the light subassembly 34 with the intermediate power cord 68. For example, to accommodate the intermediate power cord 68, a hole is drilled in the duct housing 24, and a grommet 44 is inserted into the hole. The intermediate power cord 68 is passed through the grommet 44, to electrically connect the power enclosure box 36 and the light subassembly 34.

It will be understood that, in the embodiment illustrated in FIGS. 2-3B, the activation subassembly (not shown in FIGS. 2 and 3A) preferably is mounted inside the power enclosure box 36. For example, if the activation subassembly includes the pressure-sensitive sensor subassembly “S”, then the pressure-sensitive sensor preferably is located in the power enclosure box 36. Those skilled in the art would appreciate that this would reduce the number of elements that the installer is required to install.

Various examples of arrangements of the light subassembly 34 and the power enclosure box 36 are illustrated in FIGS. 4A-4C. It will be understood that, for clarity of illustration, the side wall of the duct housing 24 and the grommet are omitted from FIG. 4C.

Any suitable means of connecting the elements of the system 20 and a power supply may be utilized. It will also be understood that in each of FIGS. 4A and 4B, preferably a quick connect connector 70 preferably is utilized, for convenient connection of the intermediate power cord 68 with a short power cord 72 extending from the light subassembly 34 (FIG. 4C). Those skilled in the art would be aware that connectors are available with a push/pull coupling mechanism which provides environmental sealing, to enable the system to be quickly installed.

As noted above, the light subassemblies 34 preferably are positioned so that UVC light emitted therefrom will affect all the air flowing through the duct 22, in order to inactivate at least a substantial portion of the infectious agents (e.g., viruses and bacteria) carried by the air. Those skilled in the art would appreciate that, in order to achieve consistent inactivation of at least a substantial portion of the infectious agents in the air flowing through the duct 22, it may be necessary or advisable to mount two or more light subassemblies 34 in proximity to each other. For example, in a duct housing with a relatively large cross-section, this arrangement may be necessary. Also, if the flow rate of the air through the duct is relatively high, more than one light subassembly may be needed, or desirable. Exemplary arrangements are shown in FIGS. 5A-5C.

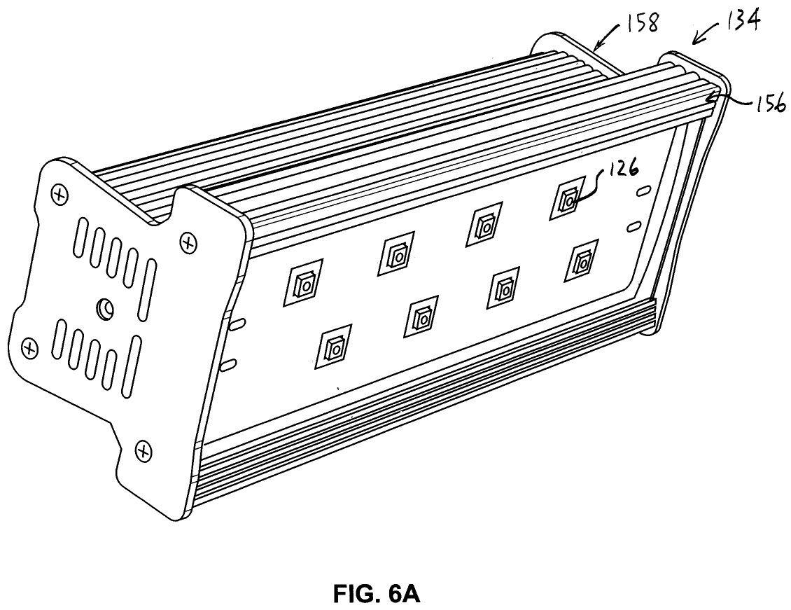

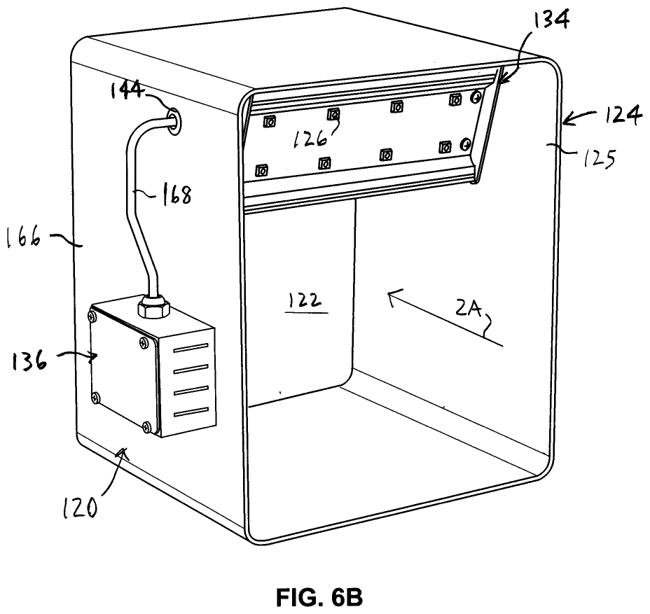

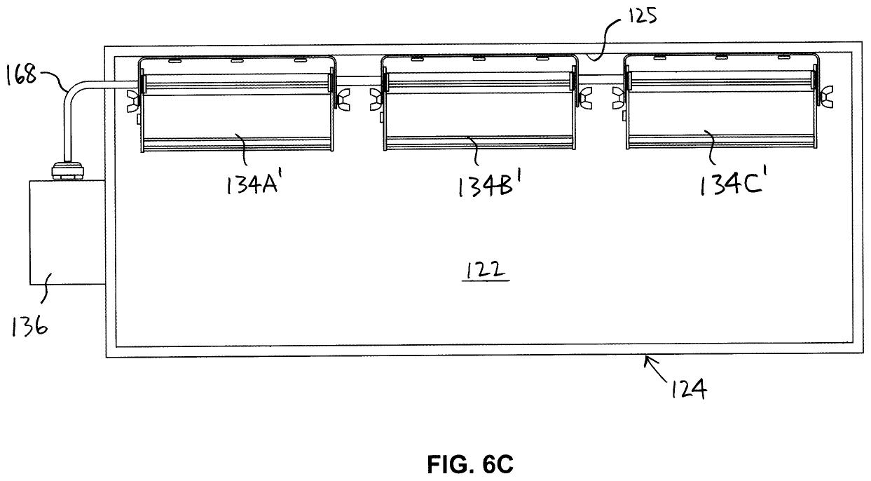

It will be understood that the embodiments of the system 20 illustrated in FIGS. 1A-5C preferably are intended for use in relatively large duct housings 24, e.g., in commercial or industrial buildings. An alternative embodiment 120 of the system of the invention, designed for use in a smaller duct housing 124, is illustrated in FIGS. 6A and 6B. For example, the system 120 may be installed in the duct housing 124 in a residential building (e.g., a single-family dwelling), in which the air flows through the duct 122 at a relatively low flow rate, as compared to the capacity of commercial systems.

As can be seen in FIG. 6B, in one embodiment, a light subassembly 134 of the system 120 preferably is mounted inside the duct housing 124, and the light subassembly 134 preferably is directly secured to one or more interior surfaces 125 of the duct housing 124. As can be seen in FIGS. 6A and 6B, the light subassembly 134 may include a relatively small number of LEDs 126. The array in the light subassembly 134 may consume 25 W.

The system 120 preferably also includes a power enclosure box 136 mounted on an exterior surface 166 of the duct housing 124. The power enclosure box 136 and the light subassembly 134 preferably are electrically connected by an intermediate power cord 168 (FIG. 6B). As can be seen in FIG. 6B, the intermediate power cord 168 preferably is located to pass through a grommet 144 mounted in the duct housing 124. In one embodiment, the power enclosure box 136 preferably is also electrically connected to an external power source (not shown).

The light subassembly 134 is designed to be easy to install inside the duct housing 124, to one or more interior surfaces 125 of the duct housing 124. The light subassembly 134 preferably includes a light housing 156 and a heat sink portion 158.

Preferably, the light subassembly 134 is mounted inside the duct housing 124 to position the LEDs 126 to emit UVC light to affect all of the air flowing through the duct 122. The direction of the air flow through the duct 122 is indicated by arrow “2A” in FIG. 6B. As illustrated, it is believed that the light subassembly 134 preferably is mounted to locate the LEDs 126 so that the UVC light emitted thereby is generally directed upstream, relative to the direction of the air flow through the duct 122. However, it will be understood that the LEDs 126 may, alternatively, be positioned so that the UVC light emitted thereby is directed downstream, or otherwise directed into the air flow.

Alternatively, the system 120′ may include more than one light subassembly, e.g., where the duct housing 124 is relatively wide. For example, as illustrated in FIG. 6C, the light subassemblies may be electrically connected to each other and mounted to the housing 124, to extend across the entire width of the housing 124. In one embodiment, the light subassemblies may consume 25 W each, and may be daisy-chained up to 100 W. For clarity of illustration, the light subassemblies are identified in FIG. 6C by reference characters 134A′, 134B′, and 134C′ respectively.

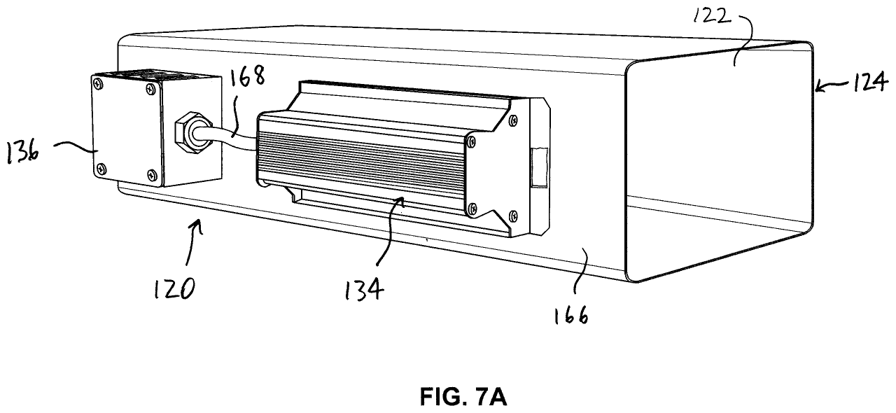

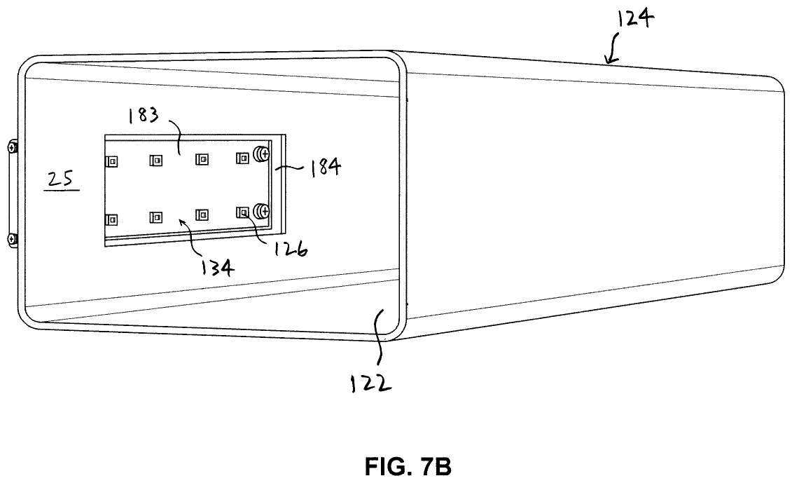

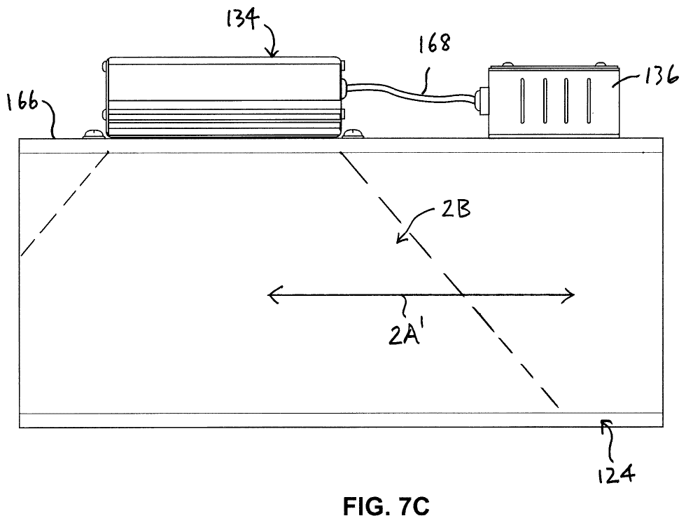

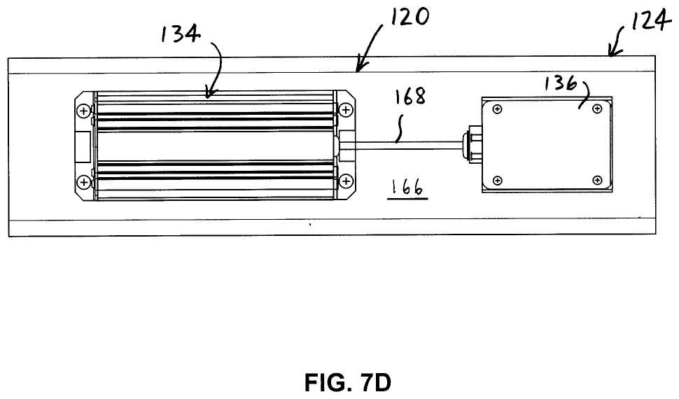

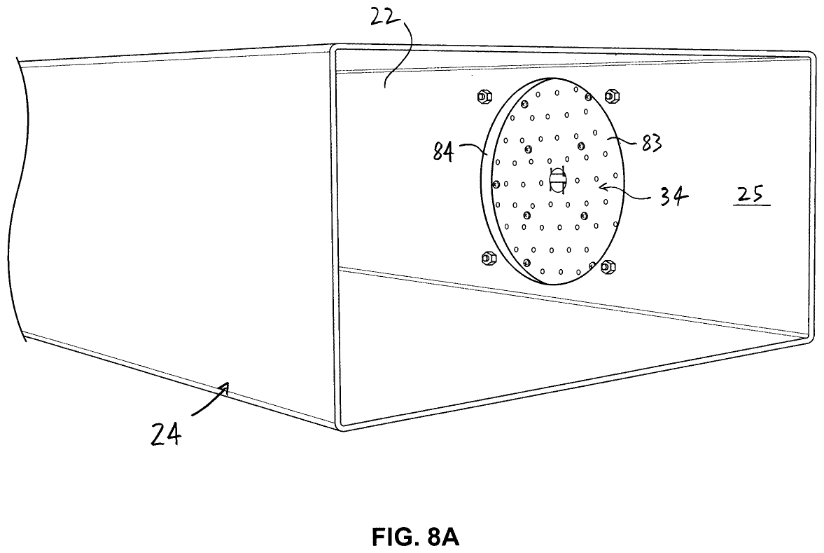

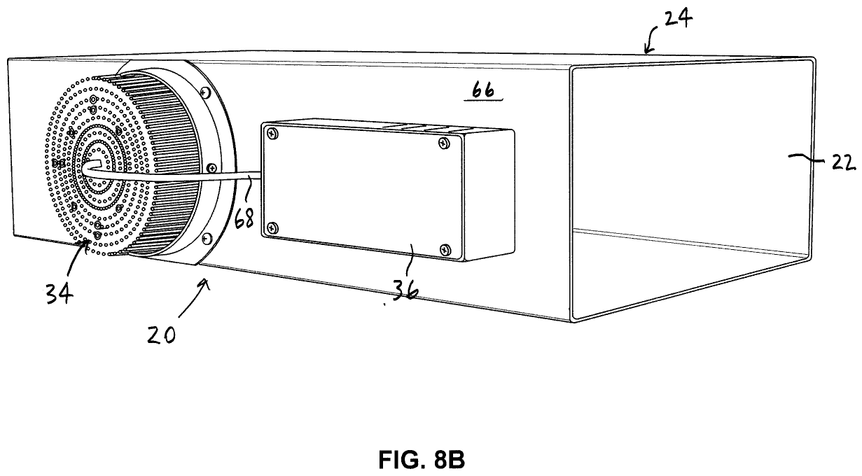

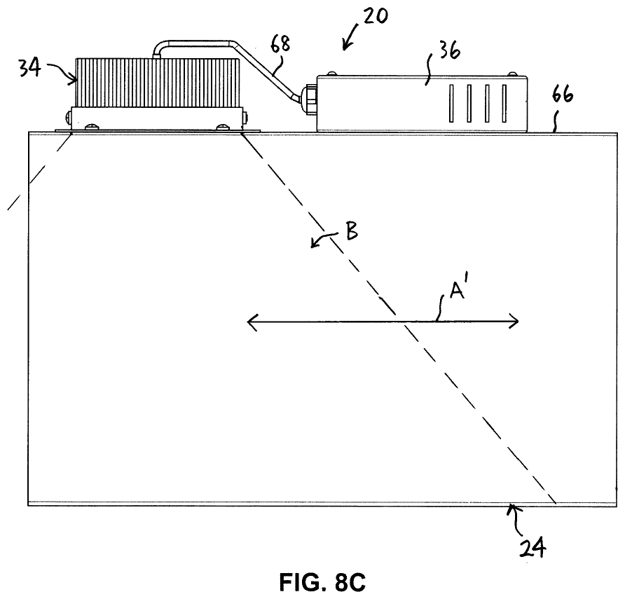

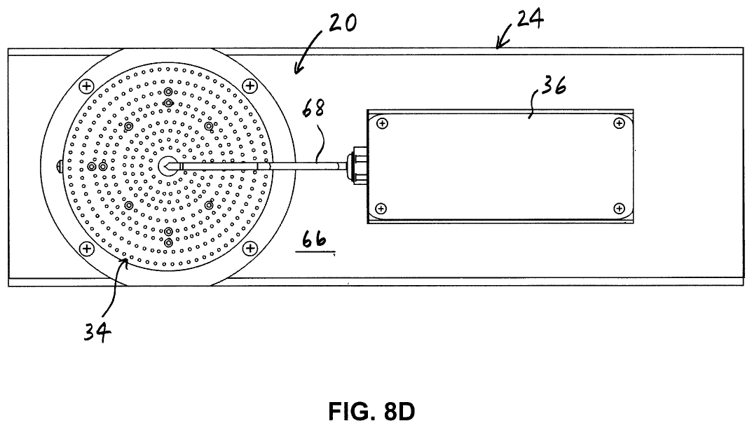

In alternative embodiments, illustrated in FIGS. 7A-8D, the light subassemblies preferably are attached to an exterior surface of the duct housing, and the UVC light therefrom is directly through a hole in the wall of the duct housing, generally transversely to the direction of air flow. These arrangements have the advantage that they may result in minimal turbulence in air flowing through the duct 22.

As can be seen in FIGS. 7A-7D, in one embodiment, the light subassembly 134 may be mounted on the exterior surface 166 of the duct housing 124. An opening 183 in the duct housing 124 is defined by an internal wall 184 (FIG. 7B). The UVC light generated by the LEDs of the light subassembly 134 is directed through the opening 183 into the duct 122, in a direction that is generally transverse to the direction of the air flow (FIGS. 7B, 7C).

The direction of the air flow is schematically indicated in FIG. 7C, by arrow 2A′. As indicated in FIG. 7C, the direction of air flow may be in either of two directions through the duct 122 (not shown in FIG. 7C). The beam of UVC light is schematically indicated by reference character “2B” in FIG. 7C.

Another arrangement is illustrated in FIGS. 8A-8D, in which the light subassembly 34 is mounted on the exterior surface 66 of the duct housing 24. In this embodiment also, the UVC light is directed into the duct 22 in a direction that is generally transverse to the direction of the air flow.

As can be seen in FIG. 8A, an opening 83 in the duct housing 24 is defined by an internal wall 84. The UVC light generated by the light subassembly 34 is directed through the opening 84 into the duct 22 (FIGS. 8A, 8C).

The direction of the air flow is schematically indicated in FIG. 8C, by arrow A′. As indicated in FIG. 8C, the direction of air flow may be in either of two directions through the duct 22 (not shown in FIG. 8C). The beam of UVC light is schematically indicated by “B” in FIG. 8C.

As is well known in the art, in a HVAC system, the air conditioning unit may contain evaporator cooling coils over which air flowing through ducts in the HVAC system pass. The cooling coils may become dirty as a result, and infectious agents may accumulate on the cooling coils. In particular, it may be that the Coronavirus may be found on cooling coils.

In one embodiment, the invention provides a system 220 for disinfecting a device 280 (e.g., cooling coils of the HVAC system) located in a duct 222 through which air flows in a predetermined direction. In FIG. 9B, the direction of air flow through the duct 222 is indicated by arrows “3A”. Preferably, the system 220 includes one or more LEDs formed for emitting UVC light.

As can be seen, e.g., in FIG. 9A, the system 220 preferably includes one or more light subassemblies 234 that are mounted in the duct 222 in a framework 282 secured inside the duct housing 224, so that the UVC light emitted from the LEDs in the light subassemblies 234 is directed toward the device 280, i.e., the cooling coils. Each of the light subassemblies 234 may be, for example, a 70 W commercial UVC LED light. It will be understood that, although UVC light is emitted from all the light subassemblies 234, for clarity of illustration, only one beam of UVC light “B” is illustrated in FIG. 9B.

As described above, the light subassemblies 234 preferably are electrically connected with a power enclosure box (not shown in FIGS. 9A-9C) for providing power to the light subassemblies 234.

It will be understood that the LEDs in the light subassembly 234 may be energized upon any suitable event occurring, in any suitable manner. For example, the light subassemblies 234 may be energized upon the fans (not shown) of the HVAC system becoming energized. This may be achieved by means of one or more switches or relays (not shown) controlling an electrical connection between a controller for the air conditioning unit and the power enclosure box. Alternatively, and as described above, the light subassembly 234 may be controlled, e.g., by a timer, or by a pressure sensor that responds to air flow in the duct 222, or by a temperature sensor.

As can be seen in FIG. 9B, in one embodiment, the light subassemblies 234 are positioned so that the UVC light generated thereby is directed downstream, relative to the direction of the air flow through the duct 222. This is preferred due to the structure of most air conditioning units, in which the cooling coils are located upstream generally relative to the balance of the air conditioning unit.

In FIG. 9C, an alternative embodiment of the system 220 is illustrated, in which a number of light subassemblies 234 are located for directing UVC light therefrom onto the device 280 (not shown in FIG. 9C).

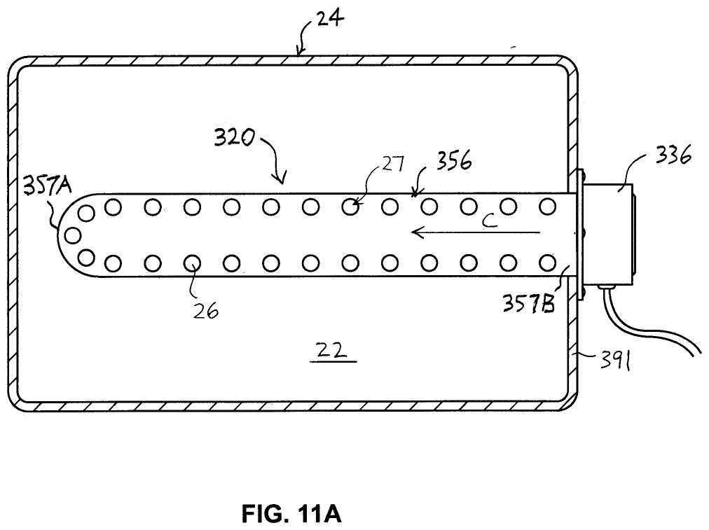

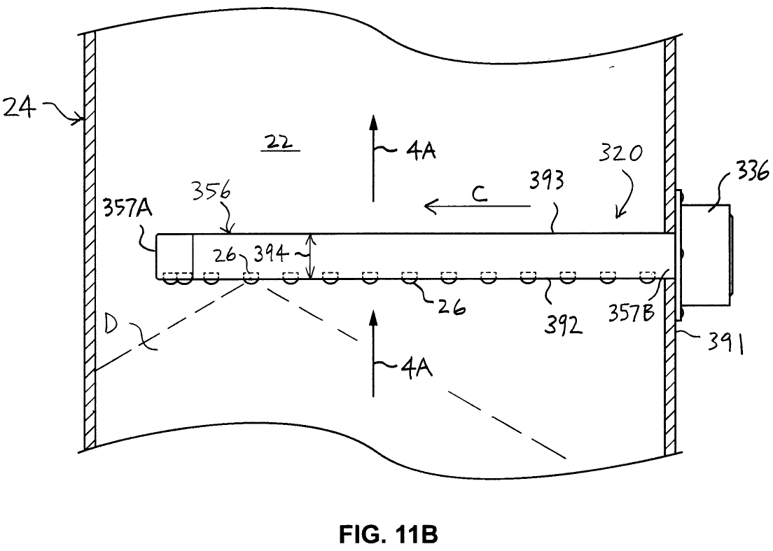

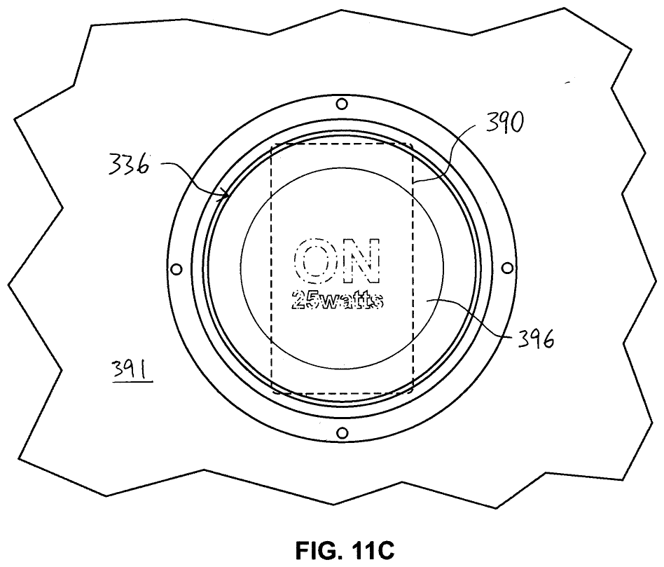

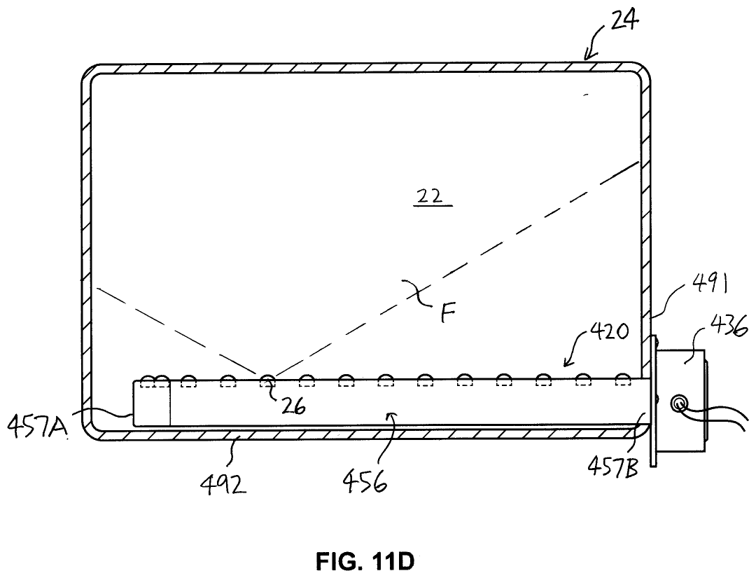

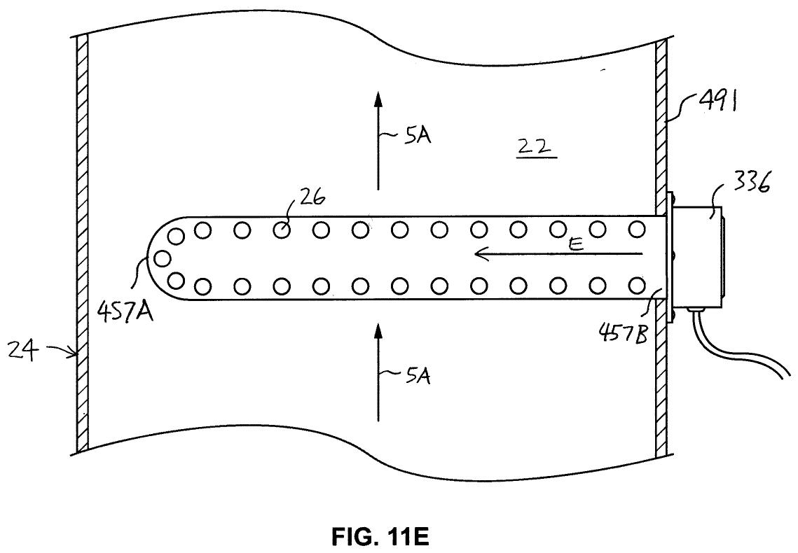

Another alternative embodiment of the system 320 of the invention is illustrated in FIGS. 11A-11C. The system 320 is designed for convenient installation in a pre-existing duct housing 24.

The system 320 preferably includes a light subassembly (not shown) that is located in an elongate light housing 356. The LEDs 26 preferably are arranged in an array 27 in the light subassembly. As can be seen in FIGS. 11A and 11B, the system 320 preferably also includes a power enclosure box 336 that is secured directly to the elongate light housing 356.

As can be seen in FIG. 11A, the light housing 356 preferably extends between a first end 357A and a second end 357B. The power enclosure box 336 is secured to the light housing 356 at the second end 357B. It will be understood that the power enclosure box 336 and the light subassembly that is inside the light housing 356 are electrically connected at the second end 357B of the light housing 356.

The light housing 356 preferably has opposed front and back sides 392, 393, which are spaced apart by a relatively narrow width 394 (FIG. 11B). Accordingly, in one embodiment, the light housing 356 is elongate, and relatively thin. It will be understood that the LEDs 26 are mounted in the light housing 356 so that the UVC light emitted thereby affects all the air moving past the light housing 356, when the light housing 356 is located in the duct 22.

In order to install the system 320, an opening 390 preferably is cut in the duct housing 24 (FIG. 11C). In the example illustrated in FIGS. 11A-11C, the opening 390 is formed in a substantially vertical sidewall 391 of the duct housing 24, however, it will be understood that the opening may alternatively be formed in one of the horizontal sidewalls. The opening 390 is formed to receive the light housing 356.

After the opening 390 has been formed, the light housing 356 may be inserted through the opening 390 and pushed into the duct 22, until the power enclosure box 336 engages the sidewall 391. To install the system 320, the first end 357A of the light housing 356 is inserted into the opening 390, and the light housing 356 is then pushed into the duct 22, in the direction indicated by arrow “C” in FIGS. 11A and 11B.

Once the light housing 356 is positioned in the duct 22, as shown in FIGS. 11A and 11B, the enclosure box 336 is secured to the sidewall 391. The enclosure box 336 may be secured to the sidewall 391 by any suitable means. For example, self-tapping screws (not shown) may be used to secure the enclosure box 336 to the sidewall 391. The power enclosure box 336 is then connected to a power source (not shown in FIGS. 11A-11C).

In one embodiment, the system 320 preferably includes a display 396 (FIG. 11C) providing basic information, e.g., the status of the system 320 (“On” or “Off”), and power consumption.

As indicated in FIGS. 11A and 11B, when energized, the LEDs 26 mounted in the light housing 356 emit UVC light into the duct 22. The UVC light from a single LED is identified for convenience in FIG. 11B by reference character “D”. The air flow direction is indicated by arrow “4A” in FIG. 11B. In FIG. 11B, the UVC light is shown as being directed upstream. However, it will be understood that the UVC light may alternatively be directed downstream inside the duct 22.

An alternative embodiment of the system 420 is illustrated in FIGS. 11C and 11D. The system 420 includes a light housing 456 and a power enclosure box 436 secured together. The light housing 456 is formed with one side flat, so that the light housing 456 may be positioned on or proximal to a horizontal bottom sidewall 492 of the duct housing 24. This arrangement may be used, for example, where positioning the light housing 456 on or proximal to the bottom sidewall 492 is convenient.

As can be seen in FIG. 11A, the light housing 456 preferably extends between a first end 457A and a second end 457B. The system 420 also includes a power enclosure box 436 that is secured to the light housing 456 at the second end 457B. It will be understood that the power enclosure box and the light subassembly (not shown) that is inside the light housing 456 are electrically connected at the second end 457B of the light housing 456.

It will be understood that, to install the system 420, a suitably sized cut or opening (not shown) is made in a sidewall 491 of the duct housing 24. The light housing 456 is then inserted into the opening and pushed in the direction indicated by arrow “E” (FIG. 11E) until the power enclosure box 436 engages the sidewall 491. The power enclosure box 436 is then secured to the sidewall 491, preferably by screws.

The LEDs 26 are arranged in an array 427 in the light housing 456 so that, when energized, the LEDs 26 direct UVC light into the air moving over the light housing 456, to at least partially disinfect the air moving through the duct 22. The UVC light from a single LED 26 is identified for convenience in FIG. 11D by reference character “F”.

The direction of air flow through the duct 22 is indicated by arrows “5A” in FIG. 11E. Accordingly, it can be seen in FIG. 11C that the UVC light is directed generally upwardly from the LEDs, generally transversely to the direction of the air flow.

From the foregoing, it can be seen that the advantage of the systems 320 and 420 is that the light housing and the power enclosure box are secured together, to provide a single physical element. Because the light housing and the power enclosure box are secured together, installation is simplified. The light housing provides a framework for positioning the LEDs therein to cause the UVC light emitted therefrom to be directed into the air in the duct 22. With this construction, the light housing can fairly easily be inserted into the duct 22 via a small opening formed in a sidewall of the duct housing 24, where convenient. The light housing is sized so that the installer only needs to push the light housing through the opening until the power enclosure box engages the exterior surface of the sidewall. Connection to the power supply is also relatively easily accomplished because the power enclosure box is secured to an exterior surface of the sidewall.

Because the systems 320, 420 are unitary, they may be sized for use in ventilation ducts that are relatively small, e.g., ventilation ducts included in vehicles. Those skilled in the art would appreciate that systems mounted in ducts in vehicles may conveniently be powered by the DC power sources in the vehicles.

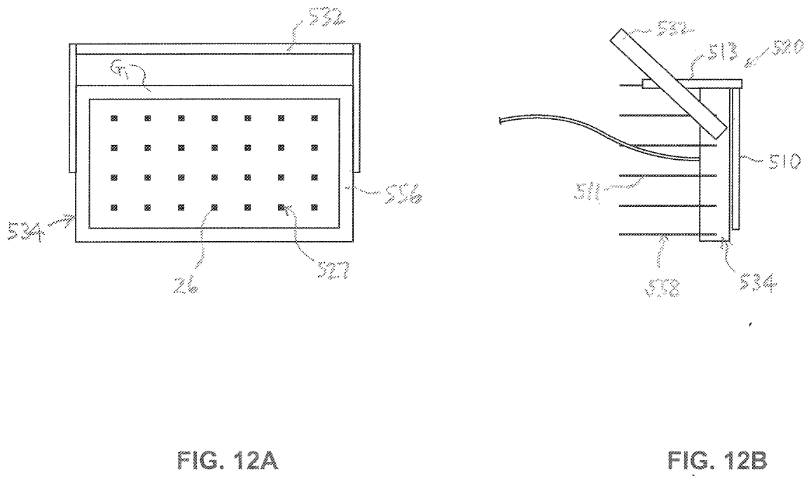

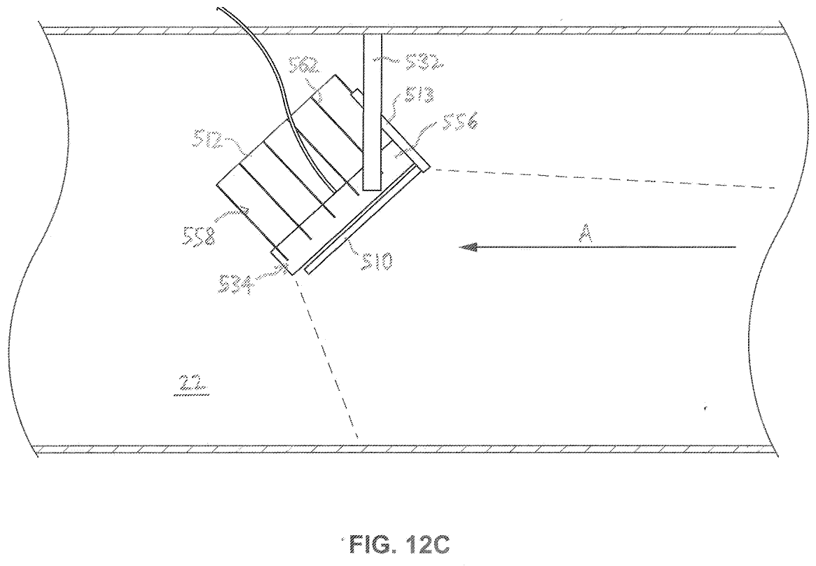

In one embodiment, the light subassembly 534 preferably includes one or more lenses 510, mounted so that the UVC light emitted by the LEDs 26 is directed through the lens 510 (FIGS. 12A-12C). For instance, a sealed quartz lens may be mounted to a light housing 556 in which an array 527 of the LEDs are mounted, to limit the extent to which moisture and contaminants in the air in the duct 22 may affect the LEDs 26.

The light housing 556 preferably includes a heat sink portion 558. As shown in FIG. 12B, the heat sink portion 558 may include a number of pins 511. Alternatively, as shown in FIG. 12C, the heat sink portion 558 may include a solid body 512 with fins 562 located thereon. The light subassembly 534 is included in an embodiment of the system 520 of the invention that preferably includes a bracket 532 for mounting the light housing 556 in the duct 22. As can be seen in FIG. 12C, the light subassembly 534 preferably is mounted for directing the UVC light emitted therefrom upstream relative to the predetermined direction of the air flow, indicated by arrow “A”.

Preferably, the light housing 556 defines a generally rectangular or square frame “G₁” in which the array 527 is located. It is believed that the shape of the light housing 556 is economically advantageous because manufacturing is thereby simplified. The system 520 preferably also includes a dust shield 513 that is for protecting the light housing 556 and the heat sink 558 from dust settling thereon, when the flow of air through the duct 22 is stopped.

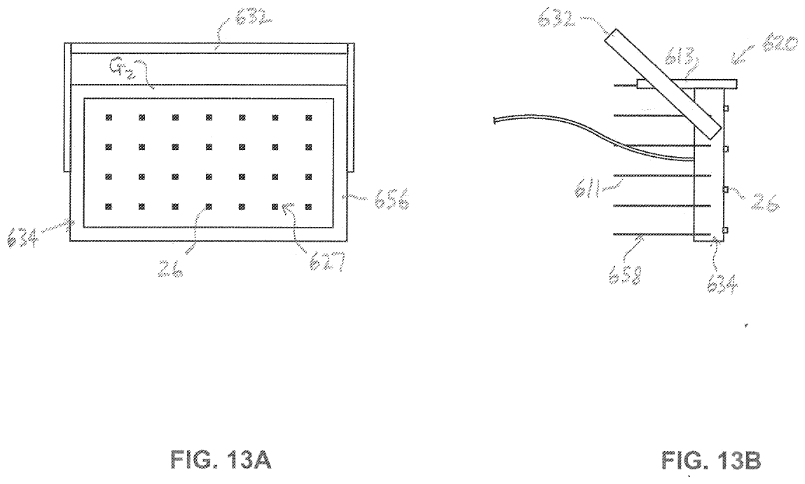

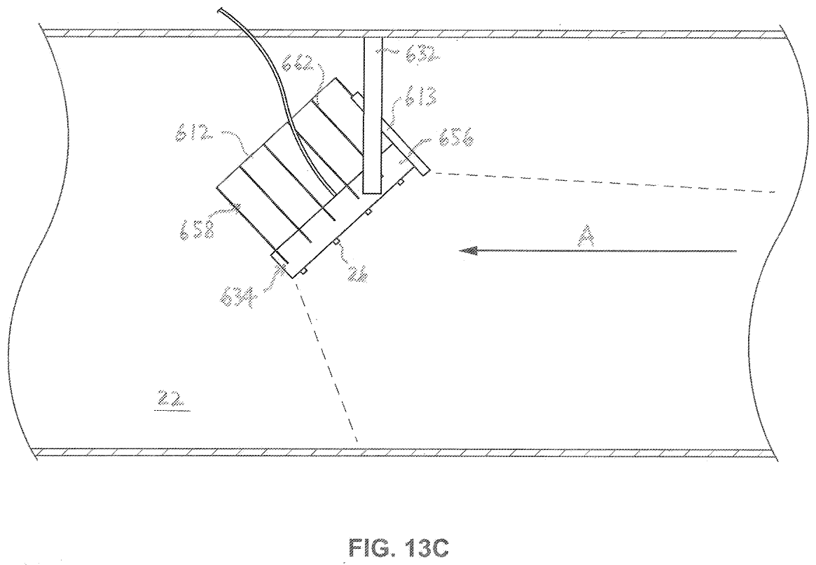

In an alternative embodiment, the light subassembly 634 may be hermetically sealed. For instance, the light subassembly 634 may include a sealant for hermetically sealing the circuit board 652 and the LEDs 26, to protect the circuit board 652 and the LEDs 26 from moisture and contaminants carried by the air in the duct (FIG. 13A-13C).

The light housing 656 preferably includes a heat sink portion 658. As shown in FIG. 13B, the heat sink portion 658 may include a number of pins 611. Alternatively, as shown in FIG. 13C, the heat sink portion 658 may include a solid body 612 with fins 662 located thereon. The light subassembly 634 is included in an embodiment of the system 620 of the invention that preferably includes a bracket 632 for mounting the light housing 656 in the duct 22. As can be seen in FIG. 13C, the light subassembly 634 preferably is mounted for directing the UVC light emitted therefrom upstream relative to the predetermined direction of the air flow, indicated by arrow “A”.

Preferably, the light housing 656 defines a generally rectangular or square frame “G₂” in which the array 627 is located. It is believed that the shape of the light housing 656 is economically advantageous because manufacturing is thereby simplified. The system 620 preferably also includes a dust shield 613 that is for protecting the light housing 656 and the heat sink 658 from dust settling thereon, when the flow of air through the duct 22 is stopped.

In one embodiment, the system 20 preferably includes sensors “AQ” for measuring selected air quality parameters, and provides for display of the measured air quality parameters. As will be described, the data from the air quality sensor “AQ” preferably is transmitted to the control subassembly 30, and the air quality data may be transmitted to the dashboard module “D”, for display of the air quality data on the display 89 (FIG. 10B).

FIG. 15A is a screen shot of exemplary air quality data for six air quality parameters of interest, showing how the air quality data may be displayed on the display 89, in one embodiment. Exemplary graphs for temperature, humidity, particulate matter, CO₂ concentration, pressure, and total VOC concentration are identified by reference characters 9A-9F respectively.

For instance, the system 20 may include one or more particulate matter detection devices (i.e., the air quality sensors “AQ” may be particulate matter detection devices) positioned for detecting an amount of particulate matter in the air in the duct 22, to provide particulate matter content data. Such data includes measured particulate matter content. In one embodiment, the particulate matter detection device preferably is configured to transmit the particulate matter content data to the control subassembly 30 for transmission to the dashboard module “D”, for displaying the measured amount of particulate matter on the display 89.

It will be understood that the control subassembly 30 preferably is configured to generate an alarm signal, transmittable to the dashboard module “D” and other preselected recipients “R”, upon the particulate matter detection device measuring particulate matter content that is greater than a preselected acceptable particulate matter content. Those informed may then take such steps as are appropriate.

In another embodiment, the system 20 preferably also includes one or more photoionization detectors (i.e., the air quality sensors “AQ” may be photoionization detectors) positioned for measuring a concentration of volatile organic compounds in the air in the duct, to provide volatile organic compound concentration data. Such data includes measured volatile organic compound concentrations. In one embodiment, the photoionization detector preferably is also configured to transmit the volatile organic compound concentration data to the control subassembly 30 for transmission to the dashboard module “D”, for displaying the concentration of volatile organic compounds on the display 89.

It will be understood that the control subassembly 30 preferably is configured to generate an alarm signal, transmittable to the dashboard module “D” and other preselected recipients “R”, upon the photoionization detector measuring volatile organic compound concentrations that are greater than preselected acceptable volatile organic compound concentrations. Those informed may then take such steps as are appropriate.

In another embodiment, the system 20 preferably also includes one or more carbon dioxide gas sensors positioned (i.e., the air quality sensors “AQ” may be carbon dioxide gas sensors) for detecting a concentration of carbon dioxide gas in the air in the duct, to provide carbon dioxide gas concentration data. Such data includes measured CO₂ concentrations. In one embodiment, the carbon dioxide gas sensor preferably is also configured to transmit the carbon dioxide gas concentration data to the control subassembly 30 for transmission to the dashboard module “D”, for displaying the concentration of carbon dioxide gas on the display 89.

It will be understood that the control subassembly 30 preferably is configured to generate an alarm signal, transmittable to the dashboard module “D” and other preselected recipients “R”, upon the carbon dioxide sensor measuring a concentration of carbon dioxide gas that is greater than a preselected acceptable carbon dioxide gas concentration. Those informed may then take such steps as are appropriate.

In another embodiment, the system 20 preferably also includes one or more bacteria level count devices (i.e., the air quality sensors “AQ” may be bacteria count level devices) positioned for evaluating microbial contamination of the air in the duct, to provide bacteria level data. In one embodiment, the bacteria level count device preferably is also configured to transmit the bacteria level data to the control subassembly 30 for transmission to the dashboard module “D”, for displaying the microbial contamination on the display 89.

It will be understood that the control subassembly 30 preferably is configured to generate an alarm signal, transmittable to the dashboard module “D” and other preselected recipients “R”, upon the bacteria level count device measuring a microbial concentration that is greater than a preselected acceptable microbial concentration. Those informed may then take such steps as are appropriate.

In an alternative embodiment of the system, the air quality sensors “AQ” may be utilized to activate the fans of the air handling unit “AHU” when the air quality parameter that is thereby measured is outside the preselected acceptable concentrations therefor. Preferably, when the fans of the air handling unit are activated, the light subassemblies 34 of the system are also activated.

For example, in one embodiment, if VOC sensors sense that the amounts of VOCs in the air passing through the duct are greater than preset acceptable concentrations thereof, then the control subassembly 30 preferably causes the fans of the air handling unit “AHU” and the light subassemblies 34 to be activated.

It is also preferred that, once the measured air quality parameters are reduced so that they are within the preselected acceptable concentrations therefor, the control subassembly 30 transmits deactivation signals to the air handling unit “AHU” and to the light subassemblies 34, and the fans of the air handling unit “AHU” and the light subassemblies 34 are deactivated.

It will also be appreciated by those skilled in the art that the invention can take many forms, and that such forms are within the scope of the invention as claimed. The scope of the claims should not be limited by the preferred embodiments set forth in the examples, but should be given the broadest interpretation consistent with the description as a whole.

We claim:

1. A system for at least partially disinfecting air movable in a predetermined direction through a duct defined by a duct housing, the air being movable through the duct by an air handling unit, the system comprising:
at least one light subassembly comprising at least one light-emitting diode formed for emitting UVC light for at least partially disinfecting the air;
a control subassembly for energizing said at least one light-emitting diode, the control subassembly comprising:
a diagnostics module for monitoring preselected parameters relating to said at least one light subassembly; and
a communications module for generating signals relating to the preselected parameters and for transmitting the signals to at least one preselected recipient thereof.

2. A system according to claim 1 additionally comprising a bracket subassembly, for locating said at least one light subassembly to direct the UVC light emitted from said at least one light-emitting diode thereof into the air in the duct.

3. A system according to claim 1 additionally comprising:
at least one display; and
a dashboard module for displaying data relating to said at least one light subassembly and the signals relating to the preselected parameters on said at least one display.

4. A system according to claim 3 in which the control subassembly is configured for deactivating said at least one light subassembly, and for activating said at least one light subassembly.

5. A system according to claim 1 in which:
the control subassembly is configured for energizing said at least one light-emitting diode when the air moves in the duct and for de-energizing said at least one light-emitting diode when the air is stationary in the duct; and
the system additionally comprises an activation subassembly, for activating the control subassembly to cause the control subassembly to energize said at least one light-emitting diode, and for deactivating the control subassembly to cause the control subassembly to de-energize said at least one light-emitting diode.

6. A system according to claim 5 in which the activation subassembly comprises:
at least one pressure sensor positioned for detecting a pressure exerted by the air flowing in the duct;
said at least one pressure sensor being configured to generate an activation signal when the pressure exerted by the air in the duct is greater than a predetermined threshold activation pressure, the activation signal being transmitted to the control subassembly for energization of said at least one light-emitting diode; and
said at least one pressure sensor being configured to generate a deactivation signal when the pressure exerted by the air in the duct is less than a predetermined threshold deactivation pressure, the deactivation signal being transmitted to the control subassembly for de-energization of said at least one light-emitting diode.

7. A system according to claim 5 in which:
the air handling unit is alternatively energized and de-energized via a relay switch operatively connected with the air handling unit, the relay switch being operable in an energizing condition, in which the air handling unit is energized, and in a de-energized condition, in which the air handling unit is de-energized; and
the activation subassembly is electrically connected with the relay switch, wherein the activation subassembly causes the control subassembly to energize said at least one light-emitting diode upon energization of the air handling unit via the relay switch, and the activation subassembly causes the control subassembly to de-energize said at least one light-emitting diode upon de-energization of the air handling unit via the relay switch.

8. A system according to claim 5 in which the activation subassembly comprises:
at least one temperature sensor positioned for detecting a temperature of the air in the duct;
said at least one temperature sensor being configured to generate an activation signal upon the temperature of the air in the duct being greater than a first predetermined threshold summer temperature, the activation signal being transmitted to the control subassembly for energization of said at least one light-emitting diode; and
said at least one temperature sensor being configured to generate a deactivation signal upon the temperature of the air in the duct being less than a second predetermined threshold summer temperature, the deactivation signal being transmitted to the control subassembly for de-energization of said at least one light-emitting diode.

9. A system according to claim 5 in which the activation subassembly comprises:
at least one temperature sensor positioned for detecting a temperature of the air in the duct;
said at least one temperature sensor being configured to generate an activation signal upon the temperature of the air in the duct being less than a first predetermined threshold winter temperature, the activation signal being transmitted to the control subassembly for energization of said at least one light-emitting diode; and
said at least one temperature sensor being configured to generate a deactivation signal upon the temperature of the air in the duct being greater than a second predetermined threshold winter temperature, the deactivation signal being transmitted to the control subassembly for de-energization of said at least one light-emitting diode.

10. A system according to claim 3 additionally comprising:
at least one particulate matter detection device positioned for detecting an amount of particulate matter in the air in the duct, to provide particulate matter content data; and
said at least one particulate matter detection device being configured to transmit the particulate matter content data to the dashboard module, for displaying the amount of particulate matter on said at least one display.

11. A system according to claim 3 additionally comprising:
at least one photoionization detector positioned for measuring a concentration of volatile organic compounds in the air in the duct, to provide volatile organic compound concentration data; and
said at least one photoionization detector being configured to transmit the volatile organic compound concentration data to the dashboard module, for displaying the concentration of volatile organic compounds on said at least one display.

12. A system according to claim 3 additionally comprising:
at least one carbon dioxide gas sensor positioned for detecting a concentration of carbon dioxide gas in the air in the duct, to provide carbon dioxide gas concentration data; and
said at least one carbon dioxide gas sensor being configured to transmit the carbon dioxide gas concentration data to the dashboard module, for displaying the concentration of carbon dioxide gas on said at least one display.

13. A system according to claim 3 additionally comprising:
at least one bacteria level count device positioned for evaluating microbial contamination of the air in the duct, to provide bacteria level data; and
said at least one bacteria level count device being configured to transmit the bacteria level data to the dashboard module, for displaying the microbial contamination on said at least one display.

14. A system according to claim 1 in which:
said at least one light-emitting diode comprises a plurality of light-emitting diodes forming an array of light-emitting diodes; and
the control subassembly is configured for de-energizing at least one preselected one of the light-emitting diodes in the array for a preselected off time period while a balance of the light-emitting diodes in the array, comprising the light-emitting diodes other than said at least one preselected one, is energized.

15. A system according to claim 14 in which, while the balance of the light-emitting diodes in the array is energized, each of the light-emitting diodes of the array is de-energized respectively for the preselected off time period.

16. A system according to claim 2 in which the bracket subassembly is securable inside the duct housing, to locate said at least one light subassembly at least partially inside the duct housing.

17. A system according to claim 2 in which the bracket subassembly is at least partially securable to an exterior surface of the duct housing.

18. A system according to claim 1 in which said at least one light subassembly is positioned to direct the UVC light emitted by said at least one light-emitting diode in an upstream direction relative to the predetermined direction.

19. A system according to claim 1 in which said at least one light subassembly is positioned to direct the UVC light emitted by said at least one light-emitting diode in a downstream direction relative to the predetermined direction.

20. A method of at least partially disinfecting air movable in a predetermined direction through a duct defined by a duct housing, the method comprising:
(a) providing at least one light subassembly comprising at least one light-emitting diode formed for emitting UVC light for at least partially disinfecting the air;
(b) providing a control subassembly for energizing said at least one light-emitting diode, the control subassembly comprising:
a diagnostics module for monitoring preselected parameters relating to said at least one light subassembly;
a communications module for generating signals relating to the preselected parameters and for transmitting the signals to at least one preselected recipient thereof;
(c) locating said at least one light-emitting diode to direct the UVC light emittable thereby into the air in the duct; and
(d) with the control subassembly, energizing said at least one light-emitting diode, causing emission of the UVC light therefrom into the air in the duct.
